(19) United States
(12) Patent Application Publication
(10) Pub. No.: US 2021/0179600 A1
(43) Pub. Date: Jun. 17, 2021

(54) OXAZOLE AND THIAZOLE DERIVATIVES AS INHIBITORS OF ASK1

(71) Applicant: Hepagene Therapeutics (HK) Limited, Wan Chai (HK)

(72) Inventor: Xiaodong XU, Doylestown, PA (US)

(73) Assignee: Hepagene Therapeutics (HK) Limited, Wan Chai (HK)

(21) Appl. No.: 16/761,655

(22) PCT Filed: Oct. 31, 2018
(86) PCT No.: PCT/US2018/058576

§ 371 Date: May 5, 2020

Related U.S. Application Data
(60) Provisional application No. 62/587,622, filed on Nov. 17, 2017.

(51) Int. Cl. C07D 413/14; C07D 417/14

(52) U.S. Cl. CPC C07D 413/14; C07D 417/14

(57) ABSTRACT

The present technology is directed to compounds of formula (I), compositions thereof, and methods related to inhibition of ASKI. In particular, the present compounds and compositions may be used to treat ASK1-mediated disorders and conditions, including, e.g., fibrotic diseases and acute and chronic liver diseases, among others.

### FIELD

The present technology is directed to compounds, compositions, and methods related to inhibition of apoptosis signal regulating kinase 1 (ASK1). In particular, the present compounds and compositions may be used to treat ASK1-mediated disorders and conditions, including, e.g., fibrotic diseases, acute and chronic liver diseases and kidney diseases.

### BACKGROUND

ASK1 is a member of the mitogen-activated protein kinase family and activates c-Jun N-terminal kinase (JINK) and p38 mitogen-activated protein kinases. ASK1 contributes to the regulation of cell death, cy tokine responses, cell differentiation and immune regulation, and has been found to be involved in fibrosis, non-alcoholic steatohepatitis (NASH), cancer, diabetes, cardiovascular and neurodegenerative diseases. Therefore, inhibitors of ASK1 are important compounds for pharmaceutical application.

### SUMMARY

In one aspect, the present technology provides a compound according to Formula I:

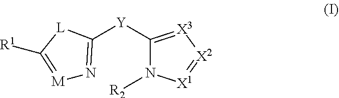

and pharmaceutically acceptable salts thereof;

wherein
L is O, NH, or S;
M is CH or N;
X¹ is CH or N;
X² is CH or N;
X³ is CH or N;
Y is a substituted or unsubstituted phenyl or 5- or 6-member heteroaryl group;
R¹ is a substituted or unsubstituted cycloalkyl, aryl or heteroaryl group; and
R² is substituted or unsubstituted alkyl, cycloalkyl, cycloalkylalkyl, aryl, or aralkyl group.

In some embodiments of compounds of Formula I, there is provided a compound of Formula IA:

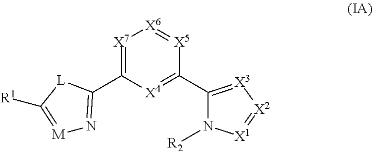

and pharmaceutically acceptable salts thereof,

wherein
X⁴ is CR⁴ or N;
X⁵ is CR⁵ or N;
X⁶ is CR⁶ or N;
X⁷ is CR⁷ or N; and
R⁴, R⁵, R⁶, and R⁺ are independently H, halo, OH, NO₂, CN, COOH, C(O)O(alkyl), C(O)O(aralkyl), C(O)O(alkenyl), C(O)(alkyl), NH₂, C(O)N₂, NH(alkyl), N(alkyl)₂, thioalkyl, haloalkyl, alkoxy, haloalkoxy, hydroxyalkyl, or a substituted or unsubstituted alkyl or cycloalkyl group; and the remaining variables (X¹, X², X³, R¹, R², L, and M) are as defined for Formula I.

In a related aspect, a composition is provided that includes any one of the compounds disclosed herein, and a pharmaceutically acceptable carrier.

In another aspect, a pharmaceutical composition is provided, the pharmaceutical composition including an effective amount of any one of the compounds disclosed herein for treating an ASK1-mediated disorder or condition.

In another aspect, a method is provided that includes administering an effective amount of any one of the compounds disclosed herein, or administering a pharmaceutical composition including an effective amount of any one of the compounds disclosed herein, to a subject suffering from an ASK1-mediated disorder or condition.

In another aspect, a method is provided that includes inhibiting ASK1 by contacting ASK1 with an effective amount of any one of the compounds of disclosed herein.

### DETAILED DESCRIPTION

In various aspects, the present technology provides compounds and methods for inhibition ASK1 and the treatment of ASK1-mediated disorders and conditions. The compounds provided herein can be formulated into pharmaceutical compositions and medicaments that are useful in the disclosed methods. Also provided is the use of the compounds in preparing pharmaceutical formulations and medicaments.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); CF₃; hydroxyls; alkoxy, alkenoxy, aryloxy, aralkyloxy, heterocyclyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; amines; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, cycloalkyl, alkenyl, alkynyl, aryl, heterocyclyl, or heteroaryl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Cycloalkenyl groups are cycloalkyl groups as defined above except that at least one double bond exists between two adjacent carbons, but the group is not aromatic, i.e., the cycloalkyl is only partially unsaturated. Exemplary monocyclic cycloalkenyl groups include, but not limited to, cyclopropenyl, cyclobutenyl, cyclopentenyl, cyclohexenyl, cycloheptenyl, and cyclooctenyl groups. In some embodiments, the cycloalkenyl group has 3 to 8 ring members, whereas in other embodiments the number of ring carbon atoms range from 3 to 5, 3 to 6, or 3 to 7. Substituted cycloalkenyl groups may be substituted one or more times with, non-hydrogen and non-carbon groups as defined above. However, substituted cycloalkenyl groups also include rings that are substituted with straight or branched chain alkyl or alkenyl groups as defined above. Representative substituted cycloalkenyl groups may be mono-substituted or substituted more than once, such as, but not limited to, 2,2-, 2,3-, 2,4-2,5- or 2,6-disubstituted cyclohexenyl groups, which may be substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons having 6-14 carbons and that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups of the present technology contain 7 to 16 carbon atoms, or in some embodiments, 7 to 14 carbon atoms, or even 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic carbon-containing ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, chromanyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic carbon containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups; divalent aryl groups are arylene groups; divalent heteroaryl groups are heteroarylene groups; and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR^{73R74} groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

With respect to substituents, the term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The phrase “selectively inhibits” as used herein will be understood by persons of ordinary skill in the art and will vary to some extent depending upon the context in which the phrase is used. If there are uses of the phrase which are not clear to persons of ordinary skill in the art, given the context in which the phrase is used, the phrase at minimum refers to the compounds acting through a specific mechanism of action, resulting in fewer off-target effects because the compounds target a particular receptor over other receptors, such as an ASK1 over other kinases. This phrase may further be modified as discussed herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, guanidines may exhibit the following isomeric forms in protic organic solution, also referred to as tautomers of each other:

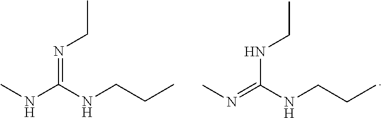

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

In one aspect, the present technology provides oxazole, imidazole, and thiazole derivatives that include pyridine, triazoles and/or other heterocycles that inhibit ASK1 and intermediates for making such compounds. The compounds include, but are not limited to compounds of Formulas I, IA, IB, IC, ID and IE as described herein.

In some aspects or embodiments of compounds of the present technology, compounds of Formula I are provided:

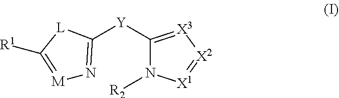

and pharmaceutically acceptable salts thereof;

wherein
L is O, NH, or S;
M is CH or N;
X¹ is CH or N;
X² is CH or N;
X³ is CH or N;
Y is a substituted or unsubstituted phenyl or a 5- or 6-member heteroaryl group;
R¹ is a substituted or unsubstituted cycloalkyl, aryl or heteroaryl group; and
R² is substituted or unsubstituted alkyl, cycloalkyl, cycloalkylalkyl, aryl, or aralkyl group.

In some embodiments of compounds of Formula I, Y is a substituted or unsubstituted 5-member heteroaryl group, e.g., thiophenyl, pyrrolyl or furan. In other embodiments, Y is a substituted or unsubstituted phenyl or 6-member heteroaryl group, e.g., pyridinyl, pyrimidinyl, or pyrazinyl. In some embodiments of compounds of Formula I, Y is a substituted or unsubstituted pyridinyl group. In others, Y is an unsubstituted pyridinyl group.

In some embodiments, there are provided compound of Formula IA:

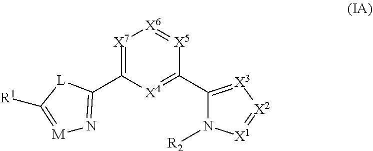

and pharmaceutically acceptable salts thereof,

wherein
X⁴ is CR⁴ or N;
X⁵ is CR⁵ or N;
X⁶ is CR⁶ or N;
X⁷ is CR⁷ or N; and
R⁴, R⁵, R⁶, and R⁷ are independently H, halo, OH, NO₂, CN, COOH, C(O)O(alkyl), C(O)O(aralkyl), C(O)O(alkenyl), C(O)(alkyl), NH₂, C(O)NH₂, NH(alkyl), N(alkyl)₂, thioalkyl, haloalkyl, alkoxy, haloalkoxy, hydroxyalkyl, or a substituted or unsubstituted alkyl or cycloalkyl group; and the remaining variables may be as defined herein.

In some embodiments of the present compounds, X¹ may be CH. In other embodiments, X¹ may be N. In some embodiments, X² may be N, and in others X² may be CH. In some embodiments X³ may be N, and in others it may be CH. In some embodiments, there are provided compounds of Formula IB:

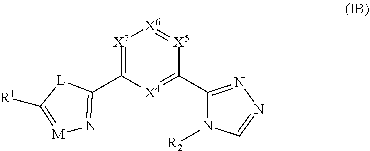

and pharmaceutically acceptable salts thereof.

In some embodiments of the present compounds, the oxazole (or imidazole or thiazole) derivative includes a substituted or unsubstituted 6-member heterocycle or phenyl group as in Formulae IA and IB. In some embodiments, R⁴, R⁵, R⁶, and R⁷ are as defined herein or are independently H, halo, OH, NH₂, or a substituted or unsubstituted alkyl or cycloalkyl group. In some embodiments of compounds herein, including but not limited to compounds of Formulae IA and IB, X⁴ may be N. In others, X⁴ may be CR⁴. In some embodiments, R⁴ is H or an unsubstituted C₁-C₆ alkyl group such a methyl. In some embodiments, X⁵ may be N. In others, X⁵ may be CR⁵. In some embodiments, R⁵ is H or an unsubstituted C₁-C₆ alkyl group such a methyl. In some embodiments, X⁶ may be N. In other embodiments, X⁶ may be CR⁶. In some embodiments, R⁶ may be H or an unsubstituted C₁-C₆ alkyl group such a methyl. In some embodiments X⁷ may be N. In others, X⁷ may be CR⁷. In some embodiments, R⁷ may be H or an unsubstituted C₁-C₆ alkyl group such a methyl.

In some embodiments, X⁴ is N, X⁵ is CR⁵, X⁶ is CR⁶, and X⁷ is CR⁷. In some embodiments, R⁵, R⁶ and R⁷ are all H. For example, the compound may be a compound of Formula IC:

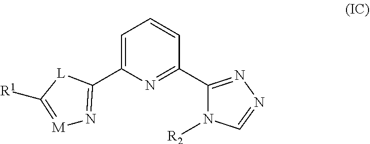

or a pharmaceutically acceptable salt thereof, wherein L, M, R¹ and are R² may be defined as in any embodiment described herein.

In some embodiments of the present compounds, L may be O, and in others L may be NH or S. In some embodiments, M may be CH and in others M may be N. In some embodiments, L is O, M is CH, and the compound has the Formula ID:

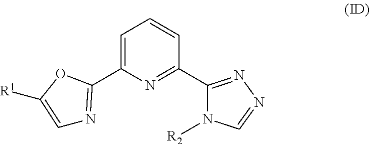

or a pharmaceutically acceptable salt thereof.

In some embodiments of the present compounds, the compound has the Formula IE:

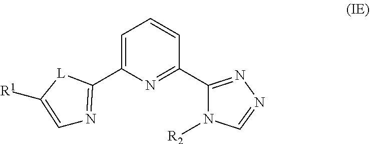

or a pharmaceutically acceptable salt thereof;

wherein L is O or S; and

R¹ and R² may be defined as in any embodiment herein.

The present compounds (including but not limited to any of the compounds of Formulas I, IA, IB, IC, ID and IE) may include a variety of R¹ groups as defined herein. In some embodiments, R¹ is a phenyl, naphthyl, tetrahydronaphthyl, cyclohexyl, pyridinyl, 2,3-dihydrobenzo[b][1,4]dioxinyl, quinolinyl, isoquinolinyl, pyrazinyl, pyrimidinyl, or oxazolyl group, optionally substituted with one or more substituents, e.g., 1, 2 or 3 substituents. In some embodiments, R¹ is phenyl, naphthyl, tetrahydronaphthyl, cyclohexyl, 2,3-dihydrobenzo[b][1,4]dioxinyl, pyridinyl, or oxazolyl, optionally substituted with one or more substituents, e.g., 1, 2 or 3 substituents. In some embodiments, W is unsubstituted. In some embodiments, R¹ is substituted with one or more substituents selected from the group consisting of F, Cl, Br, I, OH, CN, COOH, C(O)OR^{a}, C(O)R^{b}, C(O)NR^{c}R^{d}, NO₂, C(O)NH₂, NR^{e}R^{f}, SO₂NR^{g}R^{h}, alkyl, thioalkyl, haloalkyl, alkoxy, alkoxyalkoxy, haloalkoxy, hydroxyalkyl, cycloalkyl, cycloalkenyl, SO₂R^{j}, phenyl, pyrrolinyl, N-Boc-pyrrolinyl, aminopyrrolidinyl, N-Boc-aminopyrrolidinyl, pyrrolidinyl, imidazolyl, cyclopropyl-imidazolyl, oxazolyl, benzoxazolyl, thiazolyl, tetrahydro-2H-pyranyl, morpholinyl, N-alkylmorpholinyl, morpholinylalkoxy, piperidinyl, 4-morpholinyl-piperidinyl, piperazinyl, N-alkylpiperazinyl, N-cycloalkylpiperazinyl, N-sulfonylalkyl, azabicyclo-[3, 2, 1]-octanyl, and pyridinyl, wherein R^{a}, R^{b}, R^{c}, R^{d}, R^{e}, R^{f}, R^{g} and R^{h} are independently H or substituted or unsubstituted alkyl, alkenyl, cycloalkyl, aryl, aralkyl, heterocyclyl, heterocyclylalkyl, heteroaryl, or heteroaralkyl; and R^{J} is substituted or unsubstituted alkyl, alkenyl, cycloalkyl, aryl, aralkyl, heterocyclyl, heterocyclylalkyl, heteroaryl, or heteroaralkyl. In some such embodiments the alkyl groups are C₁-C₆ alkyl groups. In some such embodiments, the cycloalkyl group is a C₃-C₇ cyclalkyl group and/or the cycloalkenyl group is a C₃-C₇ cycloalkenyl group. In some embodiments, W is substituted with 1, 2, or 3 substituents wherein the substituents are selected from the group consisting of F, Cl, OH, CN, NO₂, COOH, C(O)OCH₃, C(O)R^{b}, C(O)NH₂, NH₂, C(O)NH₂, methylamino, dimethylamino, SO₂NH₂, methyl, ethyl, isopropyl, t-butyl, cyclopropyl, cyclopentyl, cyclohexyl, cycloheptyl, cyclopentenyl, cyclohexenyl, cycloheptenyl, methylthio, trifluoromethyl, difluoromethyl, methoxy, isopropoxy, n-hexyloxy, methoxyethoxy, trifluoromethoxy, difluoromethoxy, hydroxypropyl, SO₂CH₃, phenyl, pyrrolinyl, N-Boc-pyrrolinyl, 3-aminopyrrolidinyl, N-Boc-3-aminopyrrolidinyl, pyrrolidinyl, tetrahydro-2H-pyranyl, oxazolyl, benzoxazolyl, thiazolyl, cyclopropyl-imidazolyl, morpholinyl, morpholinylmethoxy, N-methyl-morpholinomethoxy, piperidinyl, 4-morpholinyl-piperidinyl, piperazinyl, N-methylpiperazinyl, N-isopropylpiperazinyl, N-cyclopropylpiperazinyl, N-sulfonylmethyl, azabicyclo-[3, 2, 1]-octanyl, and pyridinyl.

In some embodiments of the present compounds, R² may be a phenyl(C₁-C₆ alkyl), C₁-C₆ akyl or C₃-C₆ cycloalkyl group optionally substituted with one or more substituents selected from the group consisting of F, CF₃, OH, NH₂, and OCH₃. For example the phenyl(C₁-C₆ alkyl, C₁-C₆ akyl or C₃-C₆ cycloalkyl group may be substituted with one, two or three substituents. Thus, e.g., R₂ may be cyclopropyl, isopropyl, 1-hydroxy-prop-2-yl, 1,1,1-trifluropropyl, or phenylethyl.

In some embodiments, R⁴, R⁵, R⁶, and R⁷ are independently H, halo, OH, NO₂, CN, COOH, C(O)O(alkyl), C(O)O(aralkyl), C(O)O(alkenyl), C(O)(alkyl), NH₂, C(O)NH₂, NH(alkyl), N(alkyl)₂, thioalkyl, haloalkyl, alkoxy, haloalkoxy, hydroxyalkyl, or a substituted or unsubstituted alkyl, cycloalkyl group. In others, R⁴, R⁵, R⁶, and R⁷ are independently H, F, Cl, Br, OH, COOH, NH₂, or substituted or unsubstituted C₁-C₆ alkyl or C₃-C₆ cycloalkyl. In still others, R⁴, R⁵, R⁶, and R⁷ are independently H, methyl, ethyl, n-propyl, i-propyl, cyclopropyl, n-butyl, i-butyl, s-butyl, or t-butyl.

In an aspect of the present technology, a composition is provided that includes any one of the aspects and embodiments of compounds disclosed herein (e.g., compounds of Formulas (I, IA, IB, IC, ID, IE) and a pharmaceutically acceptable carrier. In a related aspect, a pharmaceutical composition is provided which includes an effective amount of the compound of any one of the aspects and embodiments of compounds of Formulas I and IA-IE for treating an ASK1-mediated disorder or condition. The ASK1-mediated disorder or condition may be fibrotic diseases including liver fibrosis, lung fibrosis, kidney fibrosis and idiopathic pulmonary fibrosis (IPF), acute and chronic liver diseases including non-alcoholic steatohepatitis (NASH), kidney diseases, autoimmune disorders, inflammatory diseases, cardiovascular diseases, diabetes, diabetic nephropathy, cardio-renal diseases, and neurodegenerative diseases. For example, the disorder or condition may be liver fibrosis or NASH.

In a further related aspect, a method is provided that includes administering an effective amount of a compound of any one of the aspects and embodiments of the present compounds or administering a pharmaceutical composition comprising an effective amount of a compound of any one of the aspects and embodiments of the present compounds to a subject suffering from an ASK1-mediated disorder or condition. The ASK1-mediated disorder or condition may be The ASK1-mediated disorder or condition may be fibrotic diseases including liver fibrosis, lung fibrosis, kidney fibrosis and IPF, acute and chronic liver diseases including NASH, kidney diseases, autoimmune disorders, inflammatory diseases, cardiovascular diseases, diabetes, diabetic nephropathy, cardio-renal diseases, and neurodegenerative diseases. For example, the disorder or condition may be liver fibrosis or NASH.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of liver fibrosis or NASH. Another example of an effective amount includes amounts or dosages that are capable of ameliorating or reducing symptoms associated with liver fibrosis or NASH. The effective amount of the compound may selectively inhibit ASK1. As used herein, a “subject” or “patient” is a mammal, such as a cat, dog, rodent or primate. Typically the subject is a human, and, preferably, a human suffering from or suspected of suffering from an ASK1-mediated disorder or condition. The term “subject” and “patient” can be used interchangeably.

In still another aspect, the present technology provides methods of inhibiting ASK1 by contacting ASK1 with an effective amount of any compound as described herein, including but not limited to a compound of Formulas I, IA, IB, IC, ID, and IE. In some embodiments, the method includes inhibiting ASK1 in vitro.

Thus, the instant present technology provides pharmaceutical compositions and medicaments comprising any of the compounds disclosed herein (e.g., compounds of Formulas I and IA-E and a pharmaceutically acceptable carrier or one or more excipients or fillers. The compositions may be used in the methods and treatments described herein. Such compositions and medicaments include a theapeutically effective amount of any compound as described herein, including but not limited to a compound of Formulas I, IA, IB, IC, ID and IE. The pharmaceutical composition may be packaged in unit dosage form.

The pharmaceutical compositions and medicaments may be prepared by mixing one or more compounds of the present technology, and/or pharmaceutically acceptable salts thereof, with pharmaceutically acceptable carriers, excipients, binders, diluents or the like to prevent and treat disorders and conditions associated with or mediated by ASK1. The compounds and compositions described herein may be used to prepare formulations and medicaments that prevent or treat a variety of disorders and conditions, including but not limited to fibrotic diseases including liver fibrosis, lung fibrosis, kidney fibrosis and IPF, acute and chronic liver diseases including NASH, kidney diseases, autoimmune disorders, inflammatory diseases, cardiovascular diseases, diabetes, diabetic nephropathy, cardio-renal diseases, and neurodegenerative diseases. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until for example, (for metabolic syndrome and/or obesity) the elevated plasma or elevated white blood cell count or hepatic cholesterol or triglycerides or progression of the disease state is reduced or stopped. For metabolic syndrome and/or obesity, the progression of the disease state can be assessed using in vivo imaging, as described, or by taking a tissue sample from a patient and observing the target of interest therein.

The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the treatment according to the present technology.

Effectiveness of the compositions and methods of the present technology may also be demonstrated by a decrease in the symptoms of hyperlipidemia, such as, for example, a decrease in triglycerides in the blood stream. Effectiveness of the compositions and methods of the present technology may also be demonstrated by a decrease in the signs and symptoms of liver disease, hyperlipidemia, hypercholesteremia, obesity, metabolic syndrome, cardiovascular disease, gastrointestinal disease, atherosclerosis, or renal disease.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of liver disease, hyperlipidemia, hypercholesteremia, obesity, metabolic syndrome, cardiovascular disease, gastrointestinal disease, atherosclerosis, or renal disease. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially be effective for the treatment of liver disease, hyperlipidemia, hypercholesteremia, obesity, metabolic syndrome, cardiovascular disease, gastrointestinal disease, atherosclerosis, or renal disease.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

The examples herein are provided to illustrate advantages of the present technology and to further assist a person of ordinary skill in the art with preparing or using the compounds of the present technology or salts, pharmaceutical compositions, derivatives, solvates, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### List of Abbreviations

ACN acetonitrile
AcOH acetic aicd
Ad₂PBu butyldi-1-adamantylphosphine
t-Bu tert-butyl
CDI 1,1′-carbonyldiimidazole
DCM dichloromethane
DIAD diisopropyl azodicarboxylate
DMF dimethylformamide
DMA dimethylacetamide
DMAP 4-dimethylaminopyridine
DMP tert-2,2-dimethoxypropane
DMSO dimethyl sulfoxide
Et ethyl
HATU (1-[bis(dimethylamino)methylene]-1H-1,2,3-triazolo[4,5-b] pyridinium 3-oxid hexafluorophosphate)
LAH lithium aluminum hydride
Me methyl
MeCN acetonitrile
NCS N-chlorosuccinimide
PCC pyridinium chlorochromate
PE petroleum ether
Ph phenyl
Py pyridine
Ruphos 2-dicyclohexylphosphino-2′,6′-diisopropoxy-1,1′-biphenyl
STAB Sodium triacetoxyborohydride
TEA triethylamine
TFA trifluoroacetic acid
TFAA trifluoroacetic anhydride
THF tetrahydrofuran
TLC thin layer chromatography
TMS trimethylsilyl
TsOH p-toluenesulfonic acid

### Example 1: Synthesis of Compound I-001

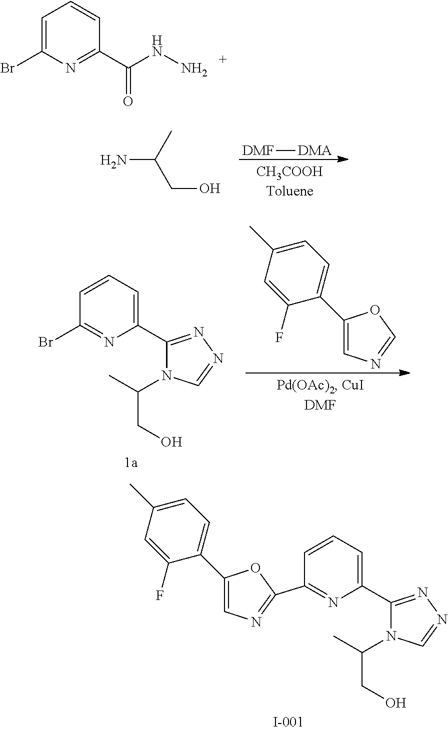

2-(3-(6-Bromopyridin-2-yl)-4H-1,2,4-triazol-4-yl) propan-1-ol (Compound 1a): To a solution of 6-bromopicolinohydrazide (200 mg, 0.93 mmol) in toluene (5 mL) was added 2-aminopropan-1-ol (500 mg, 6.67 mmol), DMF-DMA (299 mg, 2.51 mmol) and acetic acid (56 mg, 0.93 mmol). The mixture was stirred overnight at 80° C. The resulting mixture was diluted with ethyl acetate. The resulted organic phase was washed with water and brine. The residue was dried over anhydrous sodium sulfate and concentrated under vacuum. The residue was applied onto a silica gel column with dichloromethane/methanol (15:1). This resulted in 100 mg (38%) of the title compound as a white solid. LCMS (ESI, m/z): [M+H]⁺=283.0.

2-(3-(6-(5-(2-Fluoro-4-methylphenyl) oxazol-2-yl) pyridin-2-yl)-4H-1,2,4-triazol-4-yl) propan-1-ol (Compound I-001): To a solution of Compound 1a (100 mg, 0.35 mmol) and 5-(2-fluoro-4-methylphenyl) oxazole (42 mg, 0.24 mmol) in DMF (5 mL) was added Pd(OAc)₂ (2.7 mg, 0.01 mmol) and CuI (91 mg, 0.48 mmol). The mixture was stirred overnight at 140° C. under N₂ atmosphere. The resulting mixture was diluted with ethyl acetate. The resulted mixture was washed with water and brine. The residue was dried over anhydrous sodium sulfate and concentrated under vacuum. The resulted crude product was purified by Prep-HPLC with the following conditions: Column, SunFire Prep C18 OBD Column, 150 mm 5 um 10 nm; mobile phase, Water (0.1% FA) and ACN (20% ACN up to 37% in 7 min); Detector, UV 254/220 nm. This resulted in 17.2 mg (19%) of the title compound as a white solid. LCMS (ESI, m/z): [M+H]⁺=380.2. ¹HNMR (300 MHz, CD₃OD-d₄, ppm): δ 8.92 (s, 1H), 8.35-8.28 (m, 2H), 8.22-8.17 (m, 1H), 7.90-7.65 (m, 1H), 7.65 (m, 1H), 7.22-7.14 (m, 2H), 5.82 (m, 1H), 4.03-3.99 (m, 2H), 2.44 (s, 3H), 1.73-1.72 (d, J=6.9 Hz, 3H).

### Example 2: Synthesis of Compound I-002

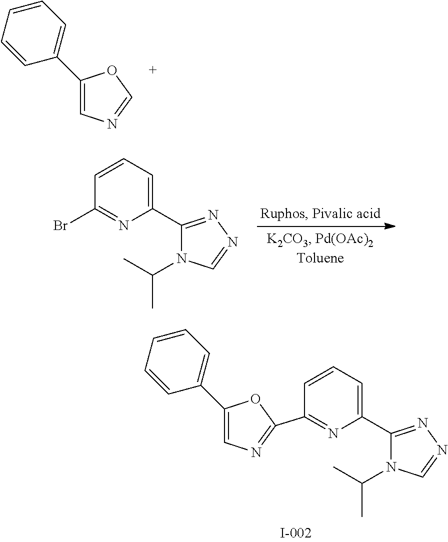

2-(5-Phenyl-1,3-oxazol-2-yl)-6-[4-(propan-2-yl)-4H-1,2,4-triazol-3-yl] pyridine (Compound I-002): To a solution of 2-bromo-6-[4-(propan-2-yl)-4H-1,2,4-triazol-3-yl]pyridine (267 mg, 1.00 mmol) in toluene (3 mL) was added 5-phenyl-1,3-oxazole (218 mg, 1.50 mmol), Ruphos (93 mg, 0.1 mmol), pivalic acid (40 mg, 0.40 mmol), potassium carbonate (414 mg, 2.97 mmol) and Pd(OAc)₂ (22.4 mg, 0.10 mmol). The resulting solution was stirred overnight at 110° C. under N₂ atmosphere. The resulting mixture was concentrated under vacuum. The residue was applied onto a silica gel column with dichloromethane/methanol (96:4). This resulted in 21 mg (6%) of the title compound as a white solid. LCMS (ESI, m/z): [M+Na]⁺=354.1. ¹HNMR (300 MHz, DMSO-d₆, ppm): δ 8.93 (s, 1H), 8.31-8.29 (m, 2H), 8.27-8.14 (m, 1H), 7.85-7.83 (m, 2H), 7.75 (s, 1H), 7.53-7.51 (m, 2H), 7.49-7.40 (m, 1H), 5.89-5.82 (m, 1H), 1.65 (s, J=6.6 Hz, 6H).

### Example 3: Synthesis of Compound I-003

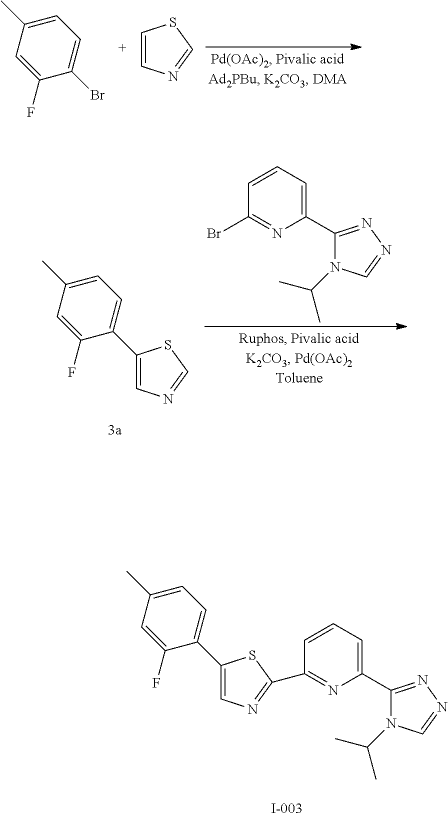

5-(2-Fluoro-4-methylphenyl)thiazole (Compound 3a): To a solution of methyl 1-bromo-2-fluoro-4-methylbenzene (378 mg, 2 mmol) in DMA (5 mL) was added thiazole (356 mg, 4 mmol), Pd(OAc)₂ (11.2 mg, 0.05 mmol), Ad₂PBu (35.8 mg, 0.10 mmol), K₂CO₃ (828 mg, 6 mmol) and pivalic acid (81.6 mg, 0.80 mmol). The mixture was stirred for 16 h at 110° C. under N₂ atmosphere. The resulting mixture was diluted with ethyl acetate. The resulted mixture was washed with water and brine. The residue was dried over anhydrous sodium sulfate and concentrated under vacuum. The residue was applied onto a silica gel column with ethyl acetate/petroleum ether (1/3, v/v). This resulted in 384 mg (99%) of the title compound as a white solid. LCMS (ESI, m/z): [M+H]⁺=194.2.

5-(2-Fluoro-4-methylphenyl)-2-(6-(4-isopropyl-4H-1,2,4-triazol-3-yl)pyridin-2-yl)thiazole (Compound I-003): To a solution of Compound 3a (67 mg, 0.25 mmol) in toluene (1.5 mL) was added 2-bromo-6-(4-isopropyl-4H-1,2,4-triazol-3-yl)pyridine (100 mg, 0.5 mmol), Pd(OAc)₂ (1.4 mg, 0.013 mmol), Ruphos (4 mg, 0.025 mmol), K₂CO₃ (103 mg, 0.75 mmol) and pivalic acid (10.2 mg, 0.10 mmol). The mixture was stirred for 16 h at 110° C. under N₂ atmosphere. The mixture was diluted with ethyl acetate and washed with water and brine. The residue was dried over anhydrous sodium sulfate and concentrated under vacuum. The residue was applied onto a silica gel column with dichloromethane/methanol (10/1, v/v). The resulted crude product was purified by Prep-HPLC with the following conditions: Column, SunFire Prep C18 OBD Column, 150 mm 5 um 10 nm; mobile phase, Water (0.1% FA) and ACN (25% ACN up to 44% in 7 min); Detector, UV 254/220 nm. This resulted in 4.8 mg (5%) of the title compound as a white solid. LCMS (ESI, m/z): [M+H]⁺=380.0. ¹HNMR (400 MHz, DMSO-d₆, ppm): δ 9.00 (s, 1H), 8.47 (s, 1H), 8.27-8.24 (m, 2H), 8.20-8.19 (m, 1H), 7.87-7.83 (m, 1H), 7.29-7.26 (m, 1H), 7.20-7.18 (m, 1H), 5.55-5.52 (m, 1H), 2.39 (s, 3H), 1.62 (d, J=6.4 Hz, 6H).

### Example 4: Synthesis of Compound I-004

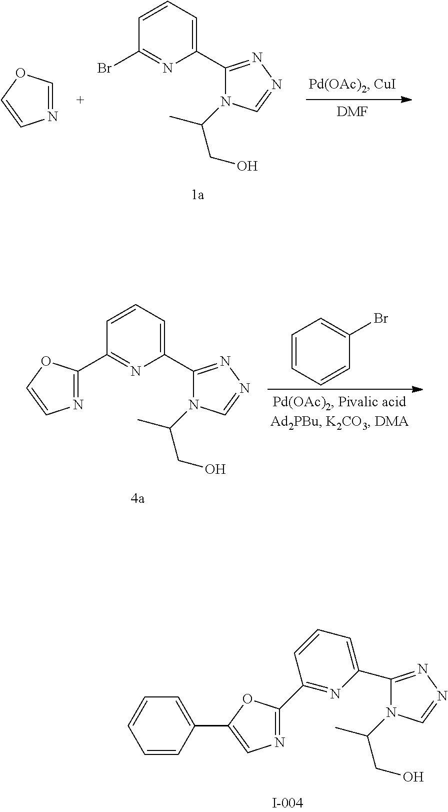

2-(3-(6-(Oxazol-2-yl) pyridin-2-yl)-4H-1,2,4-triazol-4-yl) propan-1-ol (Compound 4a): To a solution of Compound 1a (300 mg, 1.06 mmol) in DMF (2 mL) was added oxazole (49 mg, 0.71 mmol), Pd(OAc)₂ (8 mg, 0.04 mmol) and CuI (270 mg, 1.42 mmol). The mixture was stirred overnight at 140° C. under N₂ atmosphere. The resulting mixture was diluted with ethyl acetate. The resulted organic phase was washed with water and brine. The residue was dried over anhydrous sodium sulfate and concentrated under vacuum. The residue was applied onto a silica gel column with dichloromethane/methanol (10:1). This resulted in 110 mg (57%) of the title compound as a light yellow solid. LCMS (ESI, m/z): [M+H]⁺=272.0.

2-(3-(6-(5-Phenyloxazol-2-yl) pyridin-2-yl)-4H-1,2,4-triazol-4-yl) propan-1-ol (Compound I-004): To a solution of Compound 4a (110 mg, 0.43 mmol) and bromobenzene (34 mg, 0.22 mmol) in DMA (2 mL) was added Pd(OAc)₂ (2.5 mg, 0.01 mmol), Pivalic acid (9 mg, 0.40 mmol), Ad₂PBu (8 mg, 0.02 mmol) and K₂CO₃ (91 mg, 0.66 mmol). The mixture was stirred overnight at 110° C. under N₂ atmosphere. The resulting mixture was diluted with ethyl acetate. The resulted mixture was washed with water and brine. The residue was dried over anhydrous sodium sulfate and concentrated under vacuum. The resulted crude product was purified by Prep-HPLC with the following conditions: Column, SunFire Prep C18 OBD Column, 150 mm 5 um 10 nm; mobile phase, Water (0.1% FA) and ACN (27% ACN up to 47% in 7 min); Detector, UV 254/220 nm. This resulted in 14.6 mg (19%) of the title compound as a white solid. LCMS (ESI, m/z): [M+H]⁺=348.1. ¹HNMR (300 MHz, CD₃OD-d₄, ppm): δ 8.54 (s, 1H), 8.30-8.25 (m, 2H), 8.19-8.15 (m, 1H), 7.87-7.85 (m, 2H), 7.74 (s, 1H), 7.53-7.49 (m, 2H), 7.44-7.40 (m, 1H), 5.80-5.75 (m, 1H), 4.05-3.95 (m, 2H), 1.71-1.70 (d, J=3.0 Hz, 3H).

### Example 5: Synthesis of Compound I-005

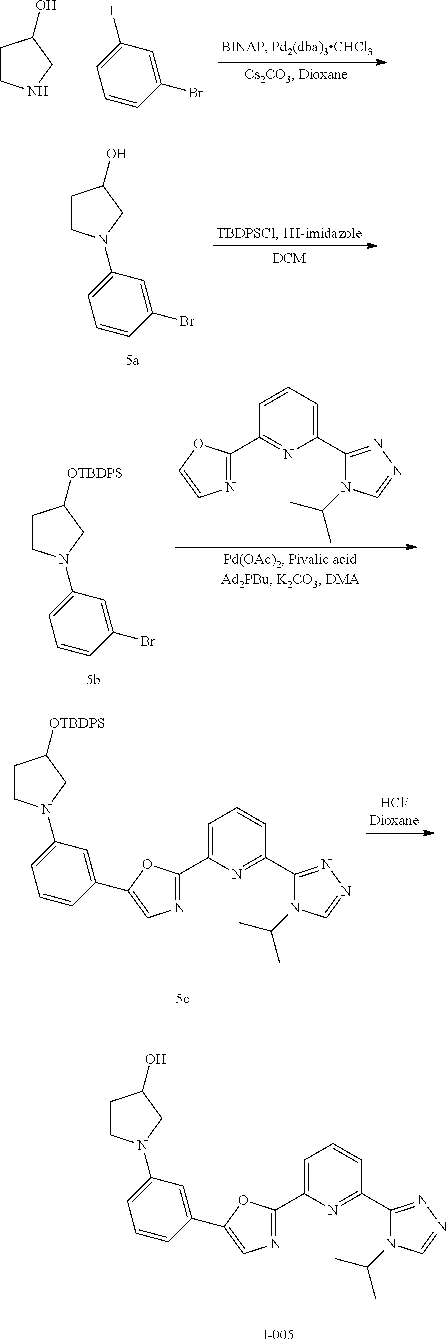

1-(3-Bromophenyl)pyrrolidin-3-ol (Compound 5a): To a mixture of 1-bromo-3-iodobenzene (500 mg, 1.77 mmol) and pyrrolidin-3-ol (154 mg, 1.77 mmol) in dioxane (10 mL) was added Cs₂CO₃ (1.7 g, 5.30 mmol), BINAP (110 mg, 0.18 mmol) and Pd₂(dba)₃.CHCl₃ (91.5 mg, 0.09 mmol). The reaction mixture was stirred at 100° C. for 3 h under N₂ atmosphere. The resulting mixture was diluted with H₂O and extracted with ethyl acetate. The organic layer was washed with brine, dried over anhydrous sodium sulfate and filtered. The filtrate was concentrated under vacuum. The residue was purified by flash column chromatography with ethyl acetate/petroleum ether (2/3, v/v) to afford the title compound (140 mg, 33%) as a yellow oil. LCMS (ESI, m/z): [M+H]⁺=242.0.

1-(3-Bromophenyl)-3-[(tert-butyldiphenylsilyl)oxy]pyrrolidine (Compound 5b): To a solution of Compound 5a (140 mg, 0.58 mmol) in DCM (7 mL) was added tert-butyl(chloro)diphenylsilane (167 mg, 0.61 mmol) and 1H-imidazole (41.3 mg, 0.61 mmol). The reaction mixture was stirred at room temperature for 16 h. The resulting mixture was diluted with ethyl acetate. The resulted organic phase was washed with brine, dried over anhydrous sodium sulfate and filtered. The filtrate was concentrated under vacuum. The residue was purified by flash column chromatography with ethyl acetate/petroleum ether (1/1, v/v) to afford the title compound (170 mg, 61%) as a yellow oil.

2-[5-(3-[3-[(tert-Butyldiphenylsilyl)oxy]pyrrolidin-1-yl]phenyl)-1,3-oxazol-2-yl]-6-[4-(propan-2-yl)-4H-1,2,4-triazol-3-yl]pyridine (Compound 5c): To a solution of Compound 5b (177 mg, 0.37 mmol) in DMA (5 mL) was added 2-(1,3-oxazol-2-yl)-6-[4-(propan-2-yl)-4H-1,2,4-triazol-3-yl]pyridine (80.0 mg, 0.31 mmol), Pd(OAc)₂ (3.5 mg, 0.02 mmol), pivalic acid (13.0 mg, 0.13 mmol), Ad₂PBu (11.2 mg, 0.03 mmol) and K₂CO₃ (130 mg, 0.94 mmol). The reaction mixture was stirred at 110° C. for 16 h under N₂ atmosphere.

The resulting mixture was diluted with H₂O and extracted with ethyl acetate. The organic layer was washed with brine, dried over anhydrous sodium sulfate and filtered. The filtrate was concentrated under vacuum. The residue was purified by flash column chromatography with dichloromethane/methanol (12/1, v/v) to afford the title compound (180 mg, 88%) as a yellow oil. LCMS (ESI, m/z): [M+H]⁺=655.3.

1-[3-(2-[6-[4-(Propan-2-yl)-4H-1,2,4-triazol-3-yl]pyridin-2-yl]-1,3-oxazol-5-yl)phenyl]pyrrolidin-3-ol (Compound I-005): To a solution of Compound 5c (180 mg, 0.28 mmol) in dioxane (5 mL) was added HCl/dioxane (5 mL, 4M). The resulting mixture was stirred at room temperature for 1 h. The mixture was concentrated under vacuum. The crude product was purified by Prep-HPLC with the following conditions: Column: XBridge Shield RP18 OBD Column 30×150 mm, 5 um; Mobile Phase A: Water (10 mmol/L NH₄HCO₃), Mobile Phase B: ACN; Flow rate: 60 mL/min; Gradient: 28% B to 50% B in 7 min; 254/220 nm; Rt: 5.98 min to afford the title compound (20.3 mg, 18%) as a yellow solid. LCMS (ESI, m/z): [M+H]⁺=417.3. ¹HNMR (300 MHz, DMSO-d₆, ppm): δ 8.99 (s, 1H), 8.28-8.16 (m, 3H), 7.96 (s, 1H), 7.34-7.29 (m, 1H), 7.10-7.07 (m, 1H), 6.92 (s, 1H), 6.61-6.57 (m, 1H), 5.70-5.61 (m, 1H), 5.02 (d, J=5.2 Hz, 1H), 4.50-4.45 (m, 1H), 3.51-3.34 (m, 3H), 3.18-3.14 (m, 1H), 2.13-2.03 (m, 1H), 1.96-1.82 (m, 1H), 1.64-1.62 (m, 6H).

### Example 6: Synthesis of Compound I-006

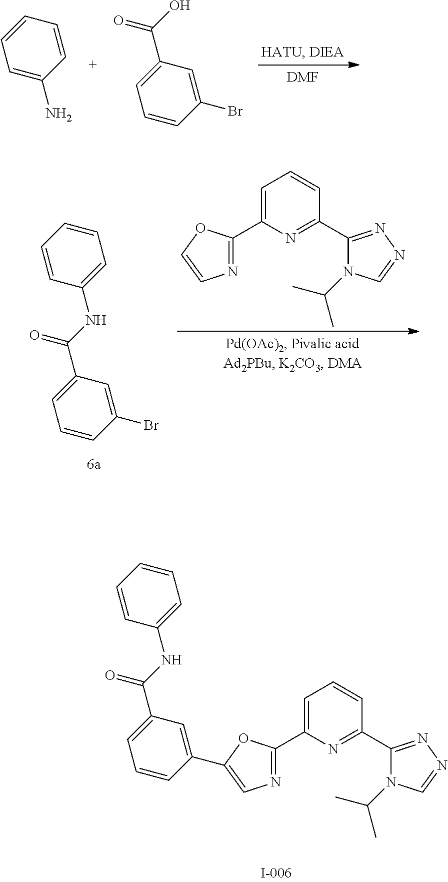

3-Bromo-N-phenylbenzamide (Compound 6a): To a solution of 3-bromobenzoic acid (1.1 g, 5.37 mmol) in DMF (25.0 mL) was added HATU (2.5 g, 6.45 mmol), DIEA (0.8 g, 6.45 mmol) at 0° C. The mixture was stirred at 0° C. for 0.5 h and then aniline (500 mg, 5.37 mmol) was added. The resulting mixture was stirred at 25° C. for 16 h. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (9/1, v/v) to afford the title compound (1.5 g, 99%) as a yellow solid. LCMS (ESI, m/z): [M+H]⁺=276.0.

N-Phenyl-3-(2-[6-[4-(propan-2-yl)-4H-1,2,4-triazol-3-yl]pyridin-2-yl]-1,3-oxazol-5-yl)benzamide (Compound I-006): To a solution of Compound 6a (108 mg, 0.39 mmol) in DMA (5.0 mL) was added 2-(1,3-oxazol-2-yl)-6-[4-(propan-2-yl)-4H-1,2,4-triazol-3-yl]pyridine (100 mg, 0.39 mmol), Pd(OAc)₂ (4.4 mg, 0.02 mmol), pivalic acid (15.9 mg, 0.16 mmol), Ad₂PBu (14.0 mg, 0.04 mmol) and K₂CO₃ (162 mg, 1.17 mmol). The reaction mixture was stirred at 110° C. for 16 h under N₂ atmosphere. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (9/1, v/v) and then purified by Prep-HPLC with the following conditions: Column: SunFire Prep C18 OBD Column 19×150 mm, Sum; Mobile Phase A:Water (0.1% FA), Mobile Phase B: ACN; Flow rate: 25 mL/min; Gradient: 36% B to 60% B in 7 min; 254, 220 nm; Rt: 6.63 min to afford the title compound (20.6 mg, 12%) as a white solid. LCMS (ESI, m/z): [M+H]⁺=451.1. ¹HNMR (300 MHz, DMSO-d₆, ppm): δ 10.44 (s, 1H), 8.97 (s, 1H), 8.40-8.35 (m, 1H), 8.33-8.30 (m, 2H), 8.28-8.23 (m, 1H), 8.21-8.06 (m, 2H), 8.01-7.98 (m, 1H), 7.83-7.75 (m, 2H), 7.72-7.69 (m, 1H), 7.42-7.37 (m, 2H), 7.17-7.12 (m, 1H), 5.84-5.75 (m, 1H), 1.64 (d, J=6.6 Hz, 6H).

### Example 7: Synthesis of Compound I-007

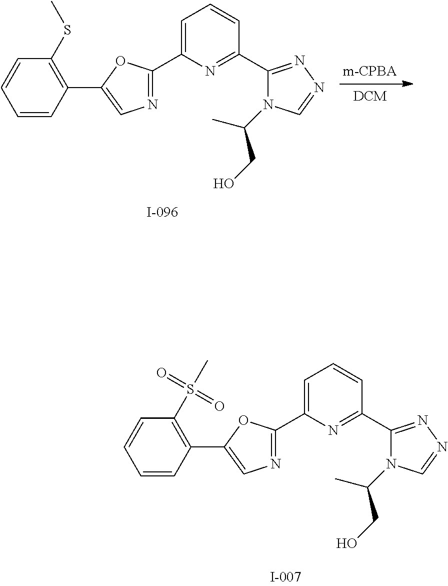 [SMILES: CSc1ccccc1-c1cnc(-c2cccc(-c3nncn3[C@H](C)CO)n2)o1 ; C[C@H](CO)n1cnnc1-c1cccc(-c2ncc(-c3ccccc3S(C)(=O)=O)o2)n1]

(2R)-2-(3-[6-[5-(2-Methanesulfonylphenyl)-1,3-oxazol-2-yl]pyridin-2-yl]-4H-1,2,4-triazol-4-yl)propan-1-ol (Compound I-007): To a solution of Compound I-096 (70.0 mg, 0.18 mmol) in DCM (1 mL) was added m-CPBA (86.7 mg, 0.50 mmol). The mixture was stirred at room temperature for 16 h. The resulting mixture was diluted with H₂O and extracted with ethyl acetate. The combined organic layer was washed with brine, dried over anhydrous sodium sulfate and filtered. The filtrate was concentrated under vacuum. The residue was purified by Prep-HPLC with the following conditions: Column: XBridge Prep OBD C18 Column 30×150 mm, 5 um; Mobile Phase A: Water (10 mmol/L NH₄HCO₃), Mobile Phase B: ACN; Flow rate: 60 mL/min; Gradient: 17% B to 32% B in 7 min; 254/220 nm; Rt: 6.68 min to afford the title compound (2.6 mg, 3%) as a white solid. LCMS (ESI, m/z): [M+H]⁺=426.1. ¹H NMR (300 MHz, DMSO-d₆, ppm): δ 8.77 (s, 1H), 8.25-8.05 (m, 4H), 7.99-7.81 (m, 3H), 7.78-7.71 (m, 1H), 5.51-5.39 (m, 1H), 4.97-4.93 (m, 1H), 3.65-3.61 (m, 2H), 3.31 (s, 3H), 1.39 (d, J=6.9 Hz, 3H).

### Example 8: Synthesis of Compound I-008

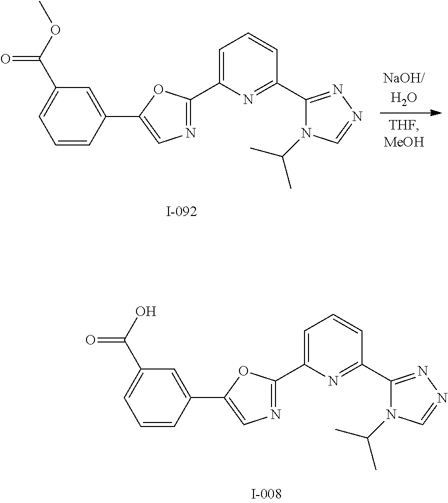 [SMILES: COC(=O)c1cccc(-c2cnc(-c3cccc(-c4nncn4C(C)C)n3)o2)c1 ; CC(C)n1cnnc1-c1cccc(-c2ncc(-c3cccc(C(=O)O)c3)o2)n1]

3-(2-[6-[4-(Propan-2-yl)-4H-1,2,4-triazol-3-yl]pyridin-2-yl]-1,3-oxazol-5-yl)benzoic acid (Compound I-008): To a solution of Compound I-092 (40.0 mg, 0.10 mmol) in a mixed solvent of methanol (2 mL) and tetrahydrofuran (2 mL) was added a solution of sodium hydroxide (21.0 mg, 0.53 mmol) in water (2 mL). The resulting mixture was stirred at 50° C. for 2 h. The pH value of the mixture was adjusted to 5 with hydrogen chloride (2 N). The mixture was concentrated under vacuum. The residue was purified by Prep-HPLC with the following conditions: Column: XBridge Prep C18 OBD Column 19×150 mm 5 um; Mobile Phase A:Water(10 mmol/L NH₄HCO₃), Mobile Phase B: ACN; Flow rate: 25 mL/min; Gradient: 32% B to 57% B in 9 min; 254, 220 nm; Rt: 6.07 min to afford the title compound (20.4 mg, 53%) as a white solid. LCMS (ESI, m/z): [M+H]⁺=376.0. ¹H NMR (400 MHz, DMSO-d₆, ppm): δ 8.99 (s, 1H), 8.41 (s, 1H), 8.33-8.31 (m, 1H), 8.25 (d, J=6.8 Hz, 1H), 8.21-8.17 (m, 1H), 8.08-8.03 (m, 1H), 7.99 (d, J=8.0 Hz, 2H), 7.66-7.62 (m, 1H), 5.88-5.81 (m, 1H), 1.68 (m, J=6.8 Hz, 6H).

### Example 9: Synthesis of Compound I-009

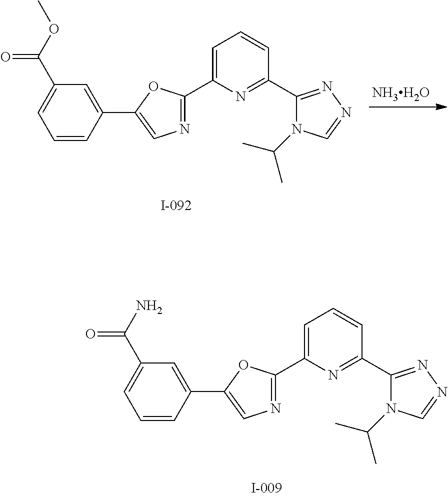 [SMILES: COC(=O)c1cccc(-c2cnc(-c3cccc(-c4nncn4C(C)C)n3)o2)c1 ; "NH3·H2O" ; CC(C)n1cnnc1-c1cccc(-c2ncc(-c3cccc(C(N)=O)c3)o2)n1]

3-(2-[6-[4-(Propan-2-yl)-4H-1,2,4-triazol-3-yl]pyridin-2-yl]-1,3-oxazol-5-yl)benzamide (Compound I-009): The solution of Compound I-092 (20 mg, 0.05 mmol) in NH₃H₂O (3 mL) was stirred at 50° C. for 16 h. The mixture was concentrated under vacuum. The residue was purified by Prep-HPLC with the following conditions: Column: XBridge Prep C18 OBD Column 19×150 mm Sum; Mobile Phase A:Water (10 mmol/L NH₄HCO₃), Mobile Phase B: ACN; Flow rate: 25 mL/min; Gradient: 8% B to 38% B in 7 min; 254, 220 nm; Rt: 4.73, 6.78 min to afford the title compound (6.7 mg, 34%) as a white solid. LCMS (ESI, m/z): [M+H]⁺=375.0. ¹H NMR (400 MHz, DMSO-d₆, ppm): δ 8.99 (s, 1H), 8.37 (s, 1H), 8.31 (d, J=7.6 Hz, 1H), 8.27 (d, J=7.2 Hz, 1H), 8.22-8.18 (m, 1H), 8.12 (s, 1H), 8.05 (s, 1H), 7.99 (d, J=7.6 Hz, 1H), 7.92 (d, J=8.0 Hz, 1H), 7.66-7.62 (m, 1H), 7.55 (s, 1H), 5.85-5.78 (m, 1H), 1.68 (d, J=6.8 Hz, 6H).

### Example 10: Synthesis of Compound I-010

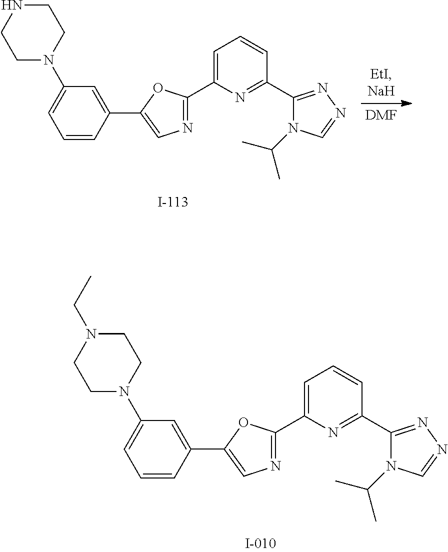

5-(3-(4-Ethylpiperazin-1-yl)phenyl)-2-(6-(4-isopropyl-4H-1,2,4-triazol-3-yl)pyridin-2-yl)oxazole (Compound I-010): To a solution of Compound I-113 (100 mg, 0.24 mmol) in DMF (3 mL) was added NaH (14.5 mg, 0.36 mmol, 60%) and iodoethane (75.0 mg, 0.48 mmol). The reaction mixture was stirred at room temperature for 4 h. After the reaction was completed, the reaction was quenched by the NH₄Cl (sat.). The reaction mixture was concentrated under vacuum. The residue was purified by Prep-HPLC with the following conditions: Column: Column: XBridge Shield RP18 OBD Column 30×150 mm, 5 um; Mobile Phase A:Water (10 mmol/L NH₄HCO₃), Mobile Phase B: ACN; Flow rate: 60 mL/min; Gradient: 28% B to 50% B in 7 min; 254/220 nm; Rt: 6.32 min to afford the title compound (10.1 mg, 10%) as a white solid. LCMS (ESI, m/z): [M+H]⁺=444.2. ¹H NMR (400 MHz, DMSO-d₆, ppm): δ 8.98 (s, 1H), 8.27-8.24 (m, 2H), 8.20-8.16 (m, 1H), 7.98 (s, 1H), 7.39-7.35 (m, 2H), 7.25 (d, J=8.0 Hz, 1H), 7.02-6.99 (m, 1H), 5.69-5.62 (m, 1H), 3.31-3.22 (m, 4H), 2.53-2.49 (m, 2H), 2.40-2.33 (m, 2H), 1.62 (d, J=6.8 Hz, 6H), 1.07-1.04 (m, 3H).

### Example 11: Synthesis of Compound I-011

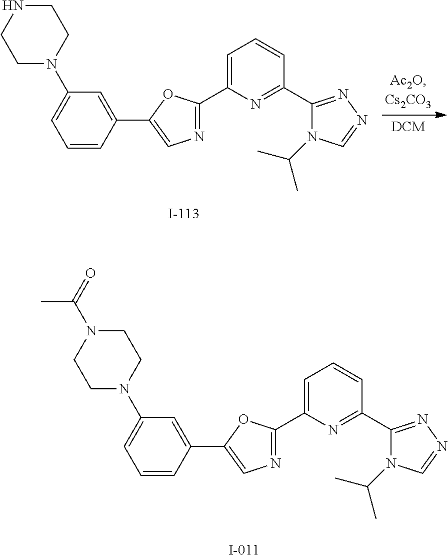

1-(4-(3-(2-(6-(4-Isopropyl-4H-1,2,4-triazol-3-yl)pyridin-2-yl)oxazol-5-yl)phenyl)piperazin-1-yl)ethenone (Compound I-011): To a solution of Compound I-113 (100 mg, 0.24 mmol) in DCM (5 mL) was added Cs₂CO₃ (156.8 mg, 0.48 mmol) and acetyl acetate (29.5 mg, 0.29 mmol). The reaction mixture was stirred at 25° C. for 3 h. The resulting mixture was diluted with DCM. The resulted organic layer was washed with brine, dried over anhydrous sodium sulfate and filtered. The filtrate was concentrated under vacuum. The residue was purified by Prep-HPLC with the following conditions: Column: XBridge Shield RP18 OBD Column 30×150 mm, 5 um; Mobile Phase A:Water (10 mmol/L NH₄HCO₃), Mobile Phase B: ACN; Flow rate: 60 mL/min; Gradient: 20% B to 48% B in 7 min; 254/220 nm; Rt: 6.07 min to afford the title compound (15.3 mg, 14%) as a white solid. LCMS (ESI, m/z): [M+H]⁺=458.2. ¹H NMR (400 MHz, DMSO-d₆, ppm): δ 8.98 (s, 1H), 8.27-8.24 (m, 2H), 8.20-8.16 (m, 1H), 7.99 (s, 1H), 7.41-7.37 (m, 2H), 7.28 (d, J=7.2 Hz, 1H), 7.05-7.02 (m, 1H), 5.68-5.61 (m, 1H), 3.62-3.60 (m, 4H), 3.30-3.18 (m, 4H), 2.06 (s, 3H), 1.63 (d, J=6.8 Hz, 6H).

### Example 12: Synthesis of Compound I-012

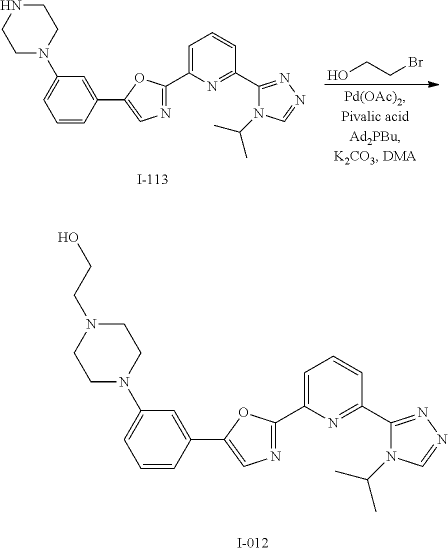

2-(4-(3-(2-(6-(4-Isopropyl-4H-1,2,4-triazol-3-yl)pyridin-2-yl)oxazol-5-yl)phenyl)piperazin-1-yl)ethanol (Compound I-012): To a solution of Compound I-113 (100 mg, 0.24 mmol) in DMA (3 mL) was added 2-bromoethan-1-ol (30.0 mg, 0.24 mmol), Pd(OAc)₂ (2.7 mg, 0.01 mmol), Ad₂PBu (8.6 mg, 0.02 mmol), K₂CO₃ (99.7 mg, 0.72 mmol) and Pivalic acid (9.8 mg, 0.09 mmol). The reaction mixture was stirred at 110° C. for 16 h under N₂ atmosphere. The resulting mixture was diluted with ethyl acetate. The resulted organic layer was washed with brine, dried over anhydrous sodium sulfate and filtered. The filtrate was concentrated under vacuum. The residue was purified by Prep-HPLC with the following conditions: Column: XBridge Prep C18 OBD Column 19×150 mm, 5 um; Mobile Phase A:Water (10 mmol/L NH₄HCO₃), Mobile Phase B: MeOH (0.1% DEA); Flow rate: 25 mL/min; Gradient: 47% B to 68% B in 7 min; 254/220 nm; Rt: 6.73 min to afford the title compound (5.2 mg, 5%) as a white solid. LCMS (ESI, m/z): [M+H]⁺=460.1. ¹H NMR (300 MHz, DMSO-d₆, ppm): δ 8.97 (s, 1H), 8.28-8.25 (m, 2H), 8.20-8.15 (m, 1H), 7.97 (s, 1H), 7.39-7.33 (m, 2H), 7.24 (d, J=8.1 Hz, 1H), 7.01-6.98 (m, 1H), 5.68-5.63 (m, 1H), 4.46-4.42 (m, 1H), 3.58-3.52 (m, 2H), 3.23-3.20 (m, 4H), 2.60-2.57 (m, 4H), 2.47-2.43 (m, 2H), 1.62 (d, J=6.9 Hz, 6H).

### Example 13: Synthesis of Compound I-013

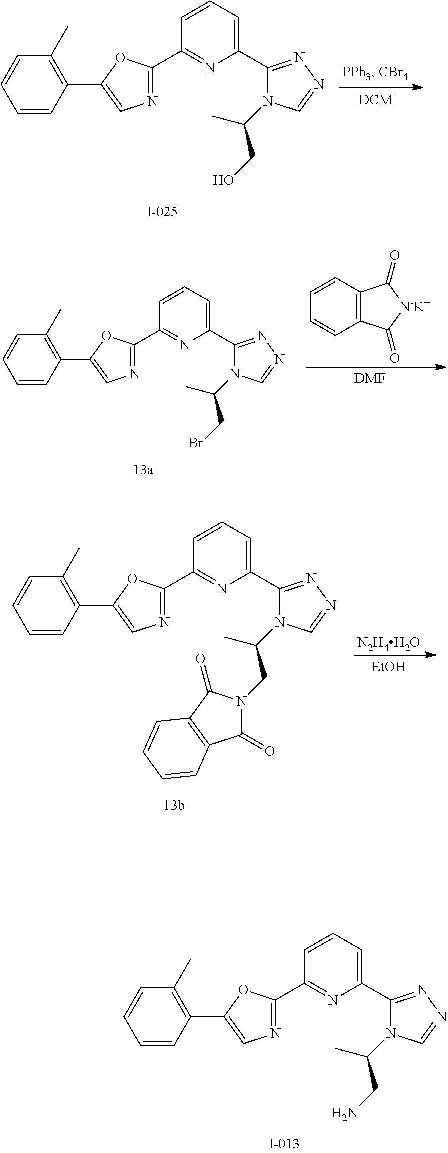

(R)-2-(6-(4-(1-Bromopropan-2-yl)-4H-1,2,4-triazol-3-yl)pyridin-2-yl)-5-o-tolyloxazole (Compound 13a): To a solution of Compound I-025 (344 mg, 0.95 mmol) in DCM (3 mL) was added CBr₄ (631 mg, 1.90 mmol) and PPh₃ (499 mg, 1.90 mmol). The mixture was stirred at room temperature for 16 h under N₂ atmosphere. The resulting mixture was concentrated under vacuum. The residue was purified by flash column chromatography with dichloromethane/methanol (9/1, v/v) to afford the title compound (200 mg, 49%) as a light green solid. LCMS (ESI, m/z): [M+H]⁺=424.0.

(R)-2-(2-(3-(6-(5-o-Tolyloxazol-2-yl)pyridin-2-yl)-4H-1,2,4-triazol-4-yl)propyl)isoindoline-1,3-dione (Compound 13b): To a solution of Compound 13a (100 mg, 0.24 mmol) in DMF (3 mL) was added potassium 1,3-dioxoisoindolin-2-ide (44.8 mg, 0.24 mmol). The reaction mixture was stirred at 90° C. for 16 h. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (9/1, v/v) to afford the title compound (100 mg, 86%) as a light yellow solid. LCMS (ESI, m/z): [M+H]⁺=491.2.

(R)-2-(3-(6-(5-o-Tolyloxazol-2-yl)pyridin-2-yl)-4H-1,2,4-triazol-4-yl)propan-1-amine (Compound I-013): To a solution of Compound 13b (100 mg, 0.20 mmol) in EtOH (3 mL) was added NH₂NH₂H₂O (20.0 mg, 0.40 mmol). The reaction mixture was stirred at 40° C. for 16 h. The mixture was concentrated under vacuum. The residue was purified by Prep-HPLC with the following conditions: Column: YMC-Actus Triart C18 30×250 mm, 5 um; Mobile Phase A:Water (10 mmol/L NH₄HCO₃), Mobile Phase B: ACN; Flow rate: 60 mL/min; Gradient: 33% B to 63% B in 7 min; 254 nm; Rt: 5.93 min to afford the title compound (18 mg, 24%) as a white solid. LCMS (ESI, m/z): [M+H]⁺=361.2. ¹H NMR (400 MHz, DMSO-d₆, ppm): δ 8.90 (s, 1H), 8.29-8.23 (m, 2H), 8.21-8.16 (m, 1H), 7.87-7.84 (m, 1H), 7.77 (d, J=2.8 Hz, 1H), 7.41-7.33 (m, 3H), 5.57-5.52 (m, 1H), 3.01-2.99 (m, 2H), 2.67 (s, 3H), 1.56 (d, J=6.8 Hz, 3H).

### Example 14: Synthesis of Compound I-014

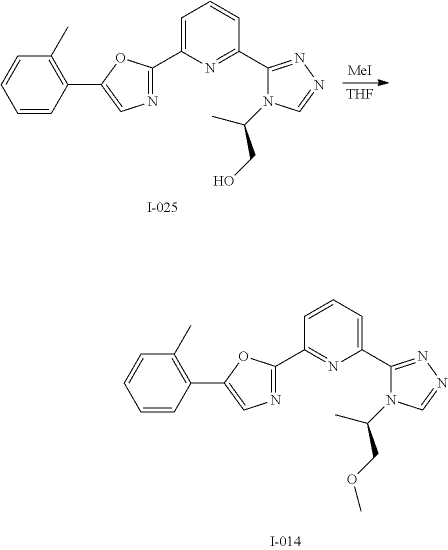 [SMILES: "MeI" ; Cc1ccccc1-c1cnc(-c2cccc(-c3nncn3[C@H](C)CO)n2)o1 ; COC[C@@H](C)n1cnnc1-c1cccc(-c2ncc(-c3ccccc3C)o2)n1 ; "THF"]

2-[4-[(2R)-1-Methoxypropan-2-yl]-4H-1,2,4-triazol-3-yl]-6-[5-(2-methylphenyl)-1,3-oxazol-2-yl]pyridine (Compound I-014): To a solution of Compound I-025 (100 mg, 0.28 mmol) in THF (4 mL) was added CH₃I (197 mg, 1.39 mmol). The reaction mixture was stirred at room temperature for 16 h. The resulting mixture was concentrated under vacuum. The residue was purified by Prep-HPLC with the following conditions: Column: SunFire Prep C18 OBD Column 19×150 mm Sum 10 nm; Mobile Phase A:Water (0.1% FA), Mobile Phase B: ACN; Flow rate: 25 mL/min; Gradient: 12% B to 30% B in 10 min; 254/220 nm; Rt: 9.00 min to afford the title compound (37.7 mg, 36%) as a white solid. LCMS (ESI, m/z): [M+H]⁺=376.2. ¹H NMR (300 MHz, DMSO-d₆, ppm): δ 8.47-8.43 (m, 2H), 8.36-8.24 (m, 2H), 7.83-7.81 (m, 2H), 7.42-7.37 (m, 3H), 5.91-5.86 (m, 1H), 4.19 (s, 3H), 4.00-3.95 (m, 1H), 3.81-3.75 (m, 1H), 2.56 (s, 3H), 1.66 (d, J=6.9 Hz, 3H).

### Example 15: Synthesis of Compound I-015

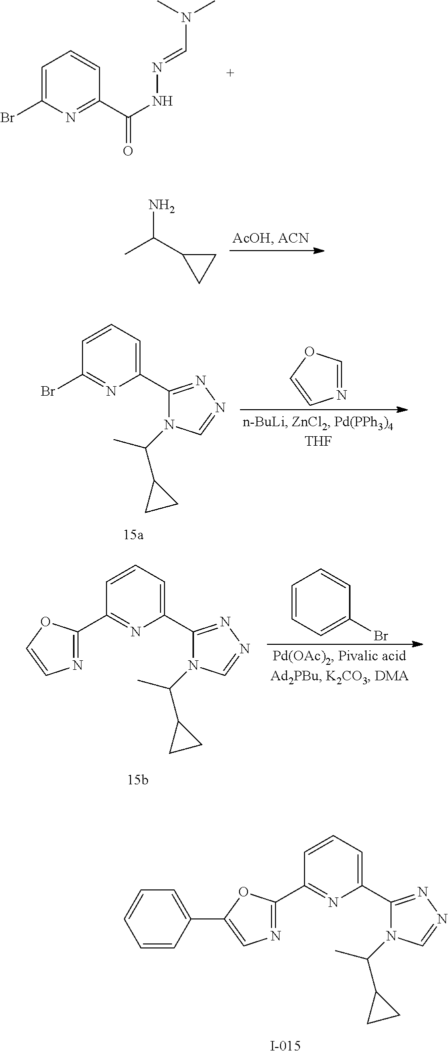

2-Bromo-6-(4-(1-cyclopropylethyl)-4H-1,2,4-triazol-3-yl)pyridine (Compound 15a): To a solution of (E)-N-(6-bromopicolinoyl)-N,N-dimethylformohydrazonamide (407 mg, 1.50 mmol) in ACN (6 mL) was added CH₃COOH (1.5 mL) at 0° C. The reaction mixture was stirred at 0° C. for 5 min. Then 1-cyclopropylethan-1-amine (908 mg, 7.50 mmol) was added to the mixture. The reaction mixture was stirred at 0° C. for another 30 min and then heated at 9° C. for 16 h. The resulting mixture was diluted with ethyl acetate. The resulted organic phase was washed with brine, dried over anhydrous sodium sulfate and filtered. The filtrate was concentrated under vacuum. The residue was purified by flash column chromatography with dichloromethane/methanol (10/1, v/v) to afford the title compound (404 mg, 92%) as a white solid. LCMS (ESI, m/z): [M+H]⁺=293.0.

2-(6-(4-(1-Cyclopropylethyl)-4H-1,2,4-triazol-3-yl)pyridin-2-yl)oxazole (Compound 15b): To a solution of oxazole (145 mg, 2.10 mmol) in THF (4 mL) was added butyllithium (0.7 mL, 1.81 mmol, 2.5 mol/L) dropwise at −78° C. under Na atmosphere. The reaction mixture was stirred at −78° C. for 30 min. Then a solution of ZnCl₂ (0.9 mL, 1.81 mmol, 2 mol/L) was added to the mixture. The reaction mixture was stirred at −78° C. for another 20 min. A mixture of Pd(PPh₃)₄ (162 mg, 0.13 mmol) and Compound 15a (404 mg, 1.38 mmol) was added to the mixture. The reaction mixture was stirred at 75° C. for 2 h under N₂ atmosphere. The resulting mixture was diluted with ethyl acetate. The resulted organic phase was washed with brine, dried over anhydrous sodium sulfate and filtered. The filtrate was concentrated under vacuum. The residue was purified by flash column chromatography with dichloromethane/methanol (10/1, v/v) to afford the title compound (328 mg, 85%) as a white solid. LCMS (ESI, m/z): [M+H]⁺=282.1.

2-(6-(4-(1-Cyclopropylethyl)-4H-1,2,4-triazol-3-yl)pyridin-2-yl)-5-phenyloxazole (Compound I-015): To a mixture of Compound 15b (84.0 mg, 0.30 mmol) and bromobenzene (71.0 mg, 0.45 mmol) in DMA (1 mL) was added Pd(OAc)₂ (2.9 mg, 0.02 mmol), Ad₂PBu (10.7 mg, 0.03 mmol), K₂CO₃ (124 mg, 0.9 mmol) and pivalic acid (24.5 mg, 0.24 mmol). The mixture was stirred at 110° C. for 16 h under N₂ atmosphere. The resulting mixture was diluted with H₂O and extracted with ethyl acetate. The resulted organic layer was washed with brine, dried over Na₂SO₄ and filtered. The filtrate was evaporated in vacuo. The residue was purified by Prep-HPLC with the following conditions: Column: SunFire Prep C18 OBD Column 19×150 mm, Sum, 10 nm; Mobile Phase A: Water (0.1% FA), Mobile Phase B: ACN; Flow rate: 25 mL/min; Gradient: 40% B to 55% B in 10 min; 254/220 nm; Rt: 9.17 min to afford the title compound (3.2 mg, 3%) as a white solid. LCMS (ESI, m/z): [M+H]⁺=358.1. ¹H NMR (400 MHz, CD₃OD-d₄, ppm): δ 9.03 (s, 1H), 8.30-8.26 (m, 2H), 8.17-8.14 (m, 1H), 7.87-7.84 (m, 2H), 7.74 (s, 1H), 7.51-7.49 (m, 2H), 7.45-7.41 (m, 1H), 5.20-5.16 (m, 1H), 1.71 (d, J=6.8 Hz, 3H), 1.59-1.55 (m, 1H), 0.83-0.79 (m, 1H), 0.64-0.51 (m, 3H).

### Example 16: Synthesis of Compound I-016

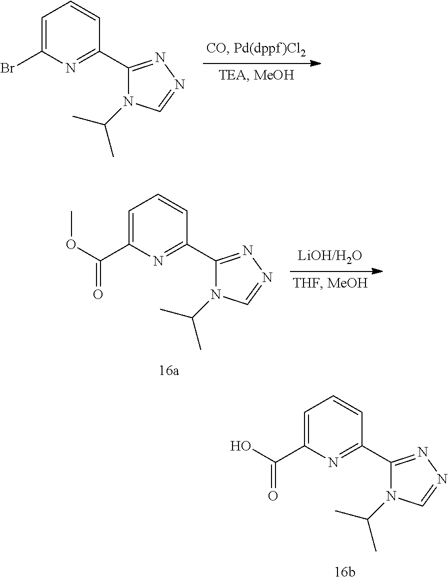

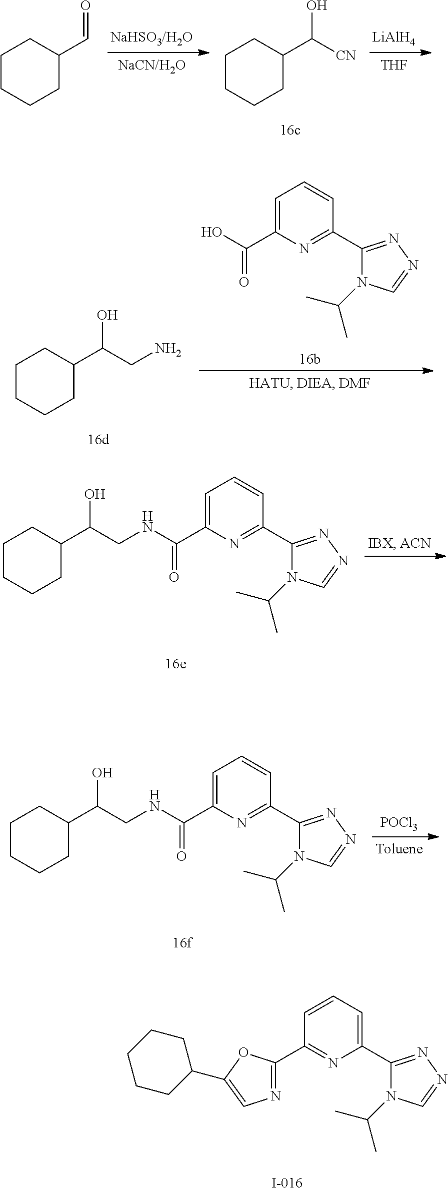

Methyl 6-(4-isopropyl-4H-1,2,4-triazol-3-yl)picolinate (Compound 16a): To a solution of 2-bromo-6-[4-(propan-2-yl)-4H-1,2,4-triazol-3-yl]pyridine (3.0 g, 11.2 mmol) in MeOH (15 mL) was added Pd(dppf)Cl₂ (1.64 g, 2.24 mmol) and TEA (3.4 g, 33.7 mmol). The reaction mixture was stirred at 70° C. for 2 h under CO (10 atm). After the reaction was completed, the reaction mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (10/1, v/v) to afford the title compound (2.8 g, 81%) as a dark grey solid. LCMS (ESI, m/z): [M+H]⁺=247.1.

6-(4-Isopropyl-4H-1,2,4-triazol-3-yl)picolinic acid (Compound 16b): To a solution of Compound 16a (2.8 g, 11.4 mmol) in MeOH (10 mL) and THF (10 mL) was added a solution of LiOH (1.36 g, 56.8 mmol) in H₂O (10 mL). The reaction mixture was stirred at 50° C. for 2 h. The pH value of the mixture was adjusted to 5 with HCl (1 mol/L). The mixture was concentrated under vacuum. The residue was purified by flash column chromatography with dichloromethane/methanol (3/1, v/v) to afford the title compound (2.0 g, 60%) as a brown solid. LCMS (ESI, m/z): [M+H]⁺=233.1.

2-Cyclohexyl-2-hydroxyacetonitrile (Compound 16c): To a solution of NaHSO₃ (2.0 g, 19.6 mmol) in H₂O (30 mL) was added cyclohexanecarbaldehyde (1.0 g, 8.92 mmol). The reaction mixture was stirred at room temperature for 1 h. Then a solution of NaCN (963 mg, 19.6 mmol) in H₂O (20 mL) was added dropwise to the mixture at 0° C. The reaction mixture was stirred at 25° C. for 16 h. The reaction mixture was diluted with EtOAc, washed with brine, dried over anhydrous sodium sulfate and filtered. The filtrate was concentrated under vacuum. The residue was purified by flash column chromatography with ethyl acetate/petroleum ether (3/1) to afford the title compound (600 mg, 38%) as a colorless oil. LCMS (ESI, m/z): [M+H]⁺=140.1.

2-Amino-1-cyclohexylethanol (Compound 16d): To a solution of Compound 16c (600 mg, 4.31 mmol) in THF (10 mL) was added LiAlH₄ (352 mg, 9.27 mmol). The resulting mixture was stirred for at 25° C. 6 h under N₂. The reaction mixture was quenched by the addition of water. The mixture was diluted with EtOAc and then filtered. The filtrate was concentrated under vacuum to afford the title compound (500 mg, crude) as a white solid. LCMS (ESI, m/z): [M+H]⁺=144.1.

N-(2-Cyclohexyl-2-hydroxyethyl)-6-(4-isopropyl-4H-1, 2, 4-triazol-3-yl)picolinamide (Compound 16e): To a solution of Compound 16b (811 mg, 3.49 mmol) in DMF (15 mL) was added HATU (1.58 g, 4.15 mmol). The mixture was stirred at 25° C. for 0.5 h. Then DIEA (1.34 g, 10.4 mmol) and Compound 16d (500 mg, 3.49 mmol) were added to the mixture. The reaction mixture was stirred at 25° C. for another 2 h. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (10/1, v/v) to afford the title compound (500 mg, 36%) as a white solid. LCMS (ESI, m/z): [M+H]⁺=358.2.

N-(2-Cyclohexyl-2-oxoethyl)-6-(4-isopropyl-4H-1, 2, 4-triazol-3-yl)picolinamide (Compound 16f): To a solution of Compound 16e (500 mg, 1.40 mmol) in CH₃CN (20 mL) was added IBX (1.18 g, 4.20 mmol) at 0° C. The reaction mixture was stirred at 70° C. for 2 h. The mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (10/1, v/v) to afford the title compound (300 mg, 57%) as a brown yellow solid. LCMS (ESI, m/z): [M+H]⁺=356.2.

5-Cyclohexyl-2-(6-(4-isopropyl-4H-1, 2, 4-triazol-3-yl) pyridin-2-yl) oxazole (Compound I-016): To a solution of Compound 16f (150 mg, 0.42 mmol) in toluene (2 mL) was added phosphoroyl trichloride (0.3 mL) at 0° C. The reaction mixture was stirred at 80° C. for 16 h under N₂ atmosphere. The resulting mixture was diluted with H₂O and extracted with ethyl acetate. The combined organic layer was washed with brine, dried over anhydrous sodium sulfate and filtered. The filtrate was concentrated under vacuum. The residue was purified by Prep-HPLC with the following conditions: Column: XBridge Prep OBD C18 Column 30×150 mm, Sum; Mobile Phase A: Water (10 mmol/L NH₄HCO₃), Mobile Phase B: ACN; Flow rate: 60 mL/min; Gradient: 34% B to 64% B in 7 min; 254/220 nm; Rt: 6.42 min to afford the title compound (18.4 mg, 12%) as a white solid. LCMS (ESI, m/z): [M+H]⁺=338.2. ¹HNMR (400 MHz, CD₃OD-d₄, ppm): δ 8.88 (s, 1H), 8.22-8.10 (m, 3H), 7.03 (s, 1H), 5.80-5.74 (m, 1H), 2.87-2.85 (m, 1H), 2.12-2.09 (m, 2H), 1.87-1.83 (m, 2H), 1.80-1.75 (m, 1H), 1.64 (d, J=6.8 Hz, 6H), 1.60-1.43 (m, 4H), 1.35-1.32 (m, 1H).

### Example 17: Synthesis of Compounds I-017 to I-144

Following the procedure described above and substituting the appropriate reagents, starting materials and purification methods known to those skilled in the art, the following compounds of the present invention were also prepared.

TABLE 1

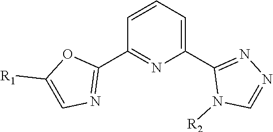

| | | | LC-MS | |
|---|---|---|---|---|
| Compound | R₁ | R₂ | (M + H)⁺ | ¹HNMR (ppm) |
| I-017 | 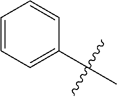 [SMILES: *c1ccccc1] | 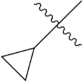 [SMILES: *C1CC1] | 330.1 | DMSO-d₆: δ 8.73 (s, 1H), 8.24-8.22 (m, 2H), 8.17-8.13 (m, 1H), 7.93 (s, 1H), 7.78 (d, J = 7.6 Hz, 2H), 7.56- 7.49 (m, 2H), 7.42-7.39 (m, 1H), 4.17- 4.11 (m, 1H), 1.17-1.05 (m, 4H). |
| I-018 | 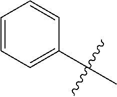 [SMILES: *c1ccccc1] | 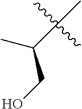 [SMILES: *[C@H](C)CO] | 348.1 | CD₃OD-d₄: δ 8.89 (s, 1H), 8.33-8.26 (m, 2H), 8.21-8.17 (m, 1H), 7.88-7.87 (m, 2H), 7.77 (s, 1H), 7.54-7.51 (m, 2H), 7.46-7.42 (m, 1H), 5.82-5.77 (m, 1H), 4.06-3.96 (m, 2H), 1.71 (d, J = 7.2 Hz, 3H). |
| I-019 | 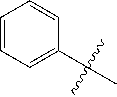 [SMILES: *c1ccccc1] | 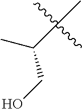 [SMILES: *[C@@H](C)CO] | 348.1 | CD₃OD-d₄: δ 8.90 (s, 1H), 8.32-8.26 (m, 2H), 8.21-8.14 (m, 1H), 7.88-7.86 (m, 2H), 7.76 (s, 1H), 7.54-7.50 (m, 2H), 7.45-7.41 (m, 1H), 5.83-5.75 (m, 1H), 4.06-3.96 (m, 2H), 1.71 (d, J = 6.8 Hz, 3H). |
| I-020 | 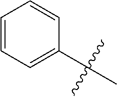 [SMILES: *c1ccccc1] | 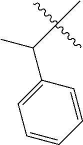 [SMILES: *C(C)c1ccccc1] | 394.1 | DMSO-d₆: δ 9.08 (s, 1H), 8.40-8.25 (m, 2H), 8.18-8.14 (m, 1H), 8.02 (s, 1H), 7.84-7.76 (m, 2H), 7.52-7.31 (m, 5H), 7.29-7.20 (m, 4H), 1.77 (d, J = 7.6 Hz, 3H). |
| I-021 | 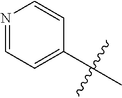 [SMILES: *c1ccncc1] | 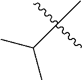 [SMILES: *C(C)C] | 333.0 | DMSO-d₆: δ 9.00 (s, 1H), 8.75-8.74 (m, 2H), 8.32-8.20 (m, 4H), 7.78-7.76 (m, 2H), 5.70-5.63 (m, 1H), 1.65 (d, J = 6.8 Hz, 6H). |
| I-022 | 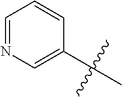 [SMILES: *c1cccnc1] | 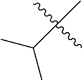 [SMILES: *C(C)C] | 333.2 | DMSO-d₆: δ 9.33 (s, 1H), 9.08-9.03 (m, 1H), 8.80-8.78 (m, 1H), 8.51-8.49 (m, 1H), 8.20-8.07 (m, 4H), 7.70-7.66 (m, 1H), 5.59-5.53 (m, 1H), 1.61-1.53 (m, 6H). |
| I-023 | 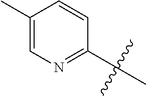 [SMILES: *c1ccc(C)cn1] | 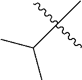 [SMILES: *C(C)C] | 347.1 | CD₃OD-d₄: δ 8.92-8.88 (m, 1H), 8.62- 8.61 (m, 1H), 8.21-8.18 (m, 4H), 7.93-7.90 (m, 2H), 5.71-5.62 (m, 1H), 2.49 (s, 3H), 1.72-1.70 (d, J = 6.0 Hz, 6H). |
| I-024 | 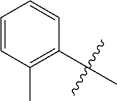 [SMILES: *c1ccccc1C] | 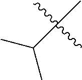 [SMILES: *C(C)C] | 346.0 | DMSO-d₆: δ 8.98 (s, 1H), 8.31-8.27 (m, 2H), 8.22-8.18 (m, 1H), 7.83-7.79 (m, 2H), 7.42-7.35 (m, 3H), 5.82-5.75 (m, 1H), 2.56 (s, 3H), 1.59 (d, J = 6.8 Hz, 6H). |
| I-025 | 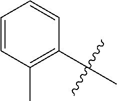 [SMILES: *c1ccccc1C] | 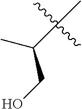 [SMILES: *[C@H](C)CO] | 362.2 | DMSO-d₆: δ 8.86 (s, 1H), 8.29-8.27 (m, 2H), 8.21-8.17 (m, 1H), 7.86-7.83 (m, 1H), 7.78 (s, 1H), 7.42-7.34 (m, 3H), 5.75-5.70 (m, 1H), 5.09-5.06 (m, 1H), 3.82-3.73 (m, 2H), 2.55 (s, 3H), 1.56 (d, J = 7.2 Hz, 3H). |
| I-026 | 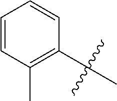 [SMILES: *c1ccccc1C] | 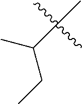 [SMILES: *C(C)CC] | 360.0 | DMSO-d₆: δ 8.97 (s, 1H), 8.31-8.17 (m, 3H), 7.83-7.78 (m, 2H), 7.42-7.34 (m, 3H), 5.73-5.66 (m, 1H), 2.52 (s, 3H), 2.00-1.86 (m, 2H), 1.56 (d, J = 6.8 Hz, 3H), 0.83-0.79 (m, 3H). |
| I-027 | 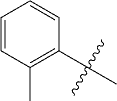 [SMILES: *c1ccccc1C] | 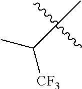 [SMILES: *C(C)C(F)(F)F] | 400.0 | DMSO-d₆: δ 9.24 (s, 1H), 8.42-8.40 (m, 1H), 8.32-8.29 (m, 1H), 8.26-8.22 (m, 1H), 7.83-7.78 (m, 2H), 7.42-7.34 (m, 3H), 7.26-7.18 (m, 1H), 2.55 (s, 3H), 1.89 (d, J = 7.2 Hz, 3H). |
| I-028 | 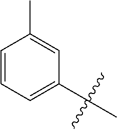 [SMILES: *c1cccc(C)c1] | 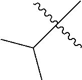 [SMILES: *C(C)C] | 346.0 | DMSO-d₆: δ 8.97 (s, 1H), 8.27-8.17 (m, 3H), 7.95 (s, 1H), 7.68-7.63 (m, 2H), 7.45-7.41 (m, 1H), 7.26 (d, J = 7.6 Hz, 1H), 5.74-5.70 (m, 1H), 2.39 (s, 3H), 1.64 (d, J = 6.8 Hz, 6H). |
| I-029 | 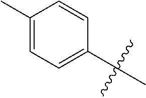 [SMILES: *c1ccc(C)cc1] | 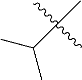 [SMILES: *C(C)C] | 346.3 | CD₃OD-d₄: δ 8.92 (s, 1H), 8.28-8.24 (m, 2H), 8.17-8.13 (m, 1H), 7.71-7.66 (m, 3H), 7.33-7.31 (m, 2H), 5.88-5.82 (m, 1H), 2.40 (s, 3H), 1.70 (d, J = 6.8 Hz, 6H). |
| I-030 | 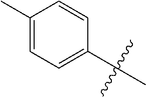 [SMILES: *c1ccc(C)cc1] | 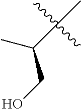 [SMILES: *[C@H](C)CO] | 362.1 | DMSO-d₆: δ 8.87 (s, 1H), 8.27-8.16 (m, 3H), 7.91 (s, 1H), 7.77-7.75 (m, 2H), 7.36 (d, J = 8.0 Hz, 2H), 5.67- 5.63 (m, 1H), 5.11-5.08 (m, 1H), 3.90- 3.70 (s, 2H), 2.37 (s, 3H), 1.59 (d, J = 6.8 Hz, 3H). |
| I-031 | 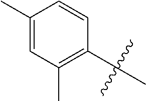 [SMILES: *c1ccc(C)cc1C] | 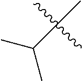 [SMILES: *C(C)C] | 360.2 | DMSO-d₆: δ 8.99 (s, 1H), 8.29-8.24 (m, 2H), 8.20-8.16 (m, 1H), 7.71-7.68 (m, 2H), 7.21-7.19 (m, 2H), 5.81-5.75 (m, 1H), 2.51 (s, 3H), 2.34 (s, 3H), 1.58 (d, J = 6.8 Hz, 6H). |
| I-032 | 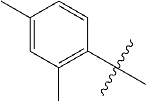 [SMILES: *c1ccc(C)cc1C] | 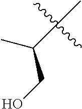 [SMILES: *[C@H](C)CO] | 376.2 | DMSO-d₆: δ 8.86 (s, 1H), 8.28-8.24 (m, 2H), 8.20-8.16 (m, 1H), 7.75-7.70 (m, 2H), 7.21-7.19 (m, 2H), 5.76-5.68 (m, 1H), 5.08-5.05 (m, 1H), 3.83-3.73 (m, 2H), 2.56 (s, 3H), 2.34 (s, 3H), 1.56 (d, J = 6.8 Hz, 3H). |
| I-033 | 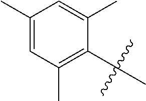 | 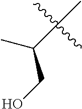 [SMILES: *[C@H](C)CO] | 390.2 | DMSO-d₆: δ 8.82 (s, 1H), 8.26-8.15 (m, 3H), 7.54 (s, 1H), 7.06-7.02 (m, 2H), 5.58-5.54 (m, 1H), 5.02-4.99 (m, 1H), 3.74-3.65 (m, 2H), 2.30 (s, 3H), 2.24 (s, 6H), 1.47 (d, J = 6.8 Hz, 3H). |
| I-034 | 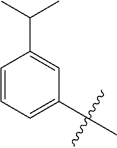 [SMILES: *c1cccc(C(C)C)c1] | 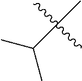 [SMILES: *C(C)C] | 374.3 | DMSO-d₆: δ 8.99 (s, 1H), 8.30-8.26 (m, 2H), 8.21-8.17 (m, 1H), 7.99 (s, 1H), 7.71-7.66 (m, 2H), 7.49-7.45 (m, 1H), 7.32 (d, J = 8.0 Hz, 1H), 5.73- 5.66 (m, 1H), 3.01-2.94 (m, 1H), 1.66- 1.61 (m, 6H), 1.28 (d, J = 6.8 Hz, 6H). |
| I-035 | 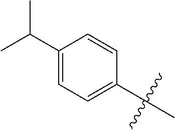 [SMILES: *c1ccc(C(C)C)cc1] | 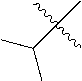 [SMILES: *C(C)C] | 374.3 | CD₃OD-d₄: δ 8.91 (s, 1H), 8.31-8.25 (m, 2H), 8.19-8.15 (m, 1H), 7.78-7.69 (m, 3H), 7.69-7.39 (m, 2H), 5.88-5.83 (m, 1H), 3.02-2.95 (m, 1H), 1.72 (d, J = 6.4 Hz, 6H), 1.30 (d, J = 6.0 Hz, 6H). |
| I-036 | 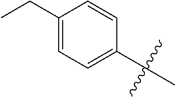 [SMILES: *c1ccc(CC)cc1] | 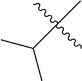 [SMILES: *C(C)C] | 360.3 | CD₃OD-d₄: δ 8.96 (s, 1H), 8.28-8.22 (m, 2H), 8.16-8.12 (m, 1H), 7.73-7.71 (m, 2H), 7.66 (s, 1H), 7.35-7.33 (m, 2H), 5.88-5.81 (m, 1H), 2.73-2.67 (m, 2H), 1.69 (d, J = 6.8 Hz, 6H), 1.28- 1.24 (m, 3H). |
| I-037 | 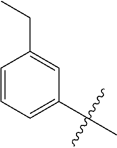 [SMILES: *c1cccc(CC)c1] | 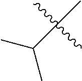 [SMILES: *C(C)C] | 360.3 | DMSO-d₆: δ 8.98 (s, 1H), 8.29-8.17 (m, 3H), 7.97 (s, 1H), 7.66 (d, J = 12.0 Hz, 2H), 7.49-7.44 (m, 1H), 7.31 (d, J = 7.5 Hz, 1H), 5.76-5.67 (m, 1H), 2.74-2.66 (m, 2H), 1.66 (d, J = 6.6 Hz, 6H), 1.28-1.23 (m, 3H). |
| I-038 | 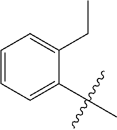 [SMILES: *c1ccccc1CC] | 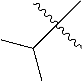 [SMILES: *C(C)C] | 360.0 | DMSO-d₆: δ 8.97 (s, 1H), 8.30-8.26 (m, 2H), 8.22-8.20 (m, 1H), 7.77-7.74 (m, 2H), 7.43-7.39 (m, 3H), 5.77-5.74 (m, 1H), 2.94-2.88 (m, 2H), 1.57 (d, J = 6.8 Hz, 6H), 1.24-1.20 (m, 3H). |
| I-039 | 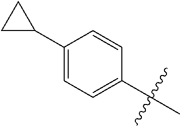 [SMILES: *c1ccc(C2CC2)cc1] | 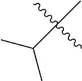 [SMILES: *C(C)C] | 372.2 | CD₃OD-d₄: δ 8.91 (s, 1H), 8.22-8.20 (m, 2H), 8.12-8.08 (m, 1H), 7.63-7.58 (m, 3H), 7.16-7.14 (m, 2H), 5.84-5.77 (m, 1H), 1.97-1.90 (m, 1H), 1.68-1.60 (m, 6H), 1.04-1.01 (m, 2H), 0.75-0.73 (m, 2H). |
| I-040 | 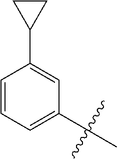 [SMILES: *c1cccc(C2CC2)c1] | 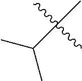 [SMILES: *C(C)C] | 372.0 | DMSO-d₆: δ 8.98 (s, 1H), 8.28-8.24 (m, 2H), 8.20-8.16 (m, 1H), 7.97 (s, 1H), 7.61 (d, J = 7.6 Hz, 1H), 7.50 (s, 1H), 7.43-7.39 (m, 1H), 7.18 (d, J = 7.6 Hz, 1H), 5.72-5.66 (m, 1H), 2.03- 1.96 (m, 1H), 1.65 (d, J = 6.8 Hz, 6H), 1.03 (d, J = 6.8 Hz, 2H), 0.77 (d, J = 4.4 Hz, 2H). |
| I-041 | 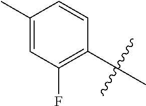 [SMILES: *c1ccc(C)cc1F] | 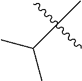 [SMILES: *C(C)C] | 364.2 | DMSO-d₆: δ 8.98 (s, 1H), 8.30-8.16 (m, 3H), 7.77-7.72 (m, 2H), 7.30-7.24 (m, 2H), 5.74-5.69 (m, 1H), 2.38 (s, 3H), 1.61 (d, J = 6.9 Hz, 6H). |
| I-042 | 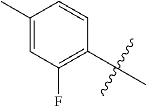 [SMILES: *c1ccc(C)cc1F] | 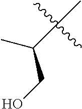 [SMILES: *[C@H](C)CO] | 380.2 | CD₃OD-d₄: δ 8.92 (s, 1H), 8.36-8.29 (m, 2H), 8.23-8.18 (m, 1H), 7.92-7.86 (m, 1H), 7.67 (d, J = 3.6 Hz, 1H), 7.23-7.16 (m, 2H), 5.88-5.81 (m, 1H), 4.03-3.99 (m, 2H), 2.45 (s, 3H), 1.72 (d, J = 6.9 Hz, 3H). |
| I-043 | 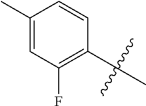 [SMILES: *c1ccc(C)cc1F] | 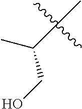 [SMILES: *[C@@H](C)CO] | 380.1 | CD₃OD-d₄: δ 8.92 (s, 1H), 8.33-8.27 (m, 2H), 8.20-8.16 (m, 1H), 7.89-7.85 (m, 1H), 7.65 (d, J = 3.6 Hz, 1H), 7.21-7.14 (m, 2H), 5.84-5.78 (m, 1H), 4.05-3.95 (m, 2H), 2.43 (s, 3H), 1.70 (d, J = 6.8 Hz, 3H). |
| I-044 | 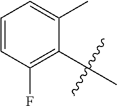 [SMILES: *c1c(C)cccc1F] | 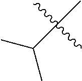 [SMILES: *C(C)C] | 364.0 | DMSO-d₆: δ 8.96 (s, 1H), 8.32-8.26 (m, 2H), 8.22-8.18 (m, 1H), 7.76 (d, J = 2.4 Hz, 1H), 7.46-7.41 (m, 1H), 7.28-7.24 (m, 2H), 5.82-5.74 (m, 1H), 2.56 (s, 3H), 1.52 (d, J = 6.4 Hz, 6H). |
| I-045 | 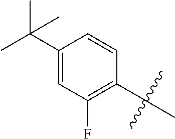 [SMILES: *c1ccc(C(C)(C)C)cc1F] | 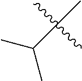 [SMILES: *C(C)C] | 406.3 | CD₃OD-d₄: δ 8.98 (s, 1H), 8.30-8.27 (m, 2H), 8.22-8.18 (m, 1H), 7.82-7.77 (m, 2H), 7.48-7.43 (m, 2H), 5.73-5.70 (m, 1H), 1.63 (d, J = 6.8 Hz, 6H), 1.33 (s, 9H). |
| I-046 | 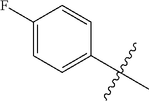 [SMILES: *c1ccc(F)cc1] | 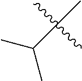 [SMILES: *C(C)C] | 350.1 | CD₃OD-d₄: δ 8.89 (s, 1H), 8.21-8.10 (m, 2H), 8.09-8.07 (m, 2H), 8.01-7.96 (m, 1H), 7.83 (s, 1H), 7.34-7.26 (m, 2H), 5.68-5.59 (m, 1H), 1.67-1.65 (d, J = 6.0 Hz, 6H). |
| I-047 | 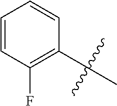 [SMILES: *c1ccccc1F] | 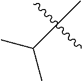 [SMILES: *C(C)C] | 350.0 | CD₃OD-d₄: δ 8.90 (s, 1H), 8.20-8.10 (m, 3H), 8.02-7.98 (m, 1H), 7.93 (s, 1H), 7.64-7.59 (m, 1H), 7.41-7.34 (m, 2H), 5.78-5.72 (m, 1H), 1.68-1.67 (d, J = 6.4 Hz, 6H). |
| I-048 | 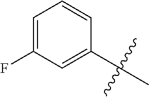 [SMILES: *c1cccc(F)c1] | 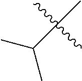 [SMILES: *C(C)C] | 350.0 | CD₃OD-d₄: δ 8.91 (s, 1H), 8.16-8.05 (m, 2H), 8.05-7.99 (m, 2H), 7.90-7.87 (m, 2H), 7.64-7.58 (m, 1H), 7.36-7.31 (m, 1H), 5.71-5.65 (m, 1H), 1.70-1.68 (d, J = 6.8 Hz, 6H). |
| I-049 | 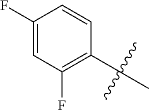 [SMILES: *c1ccc(F)cc1F] | 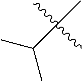 [SMILES: *C(C)C] | 367.9 | DMSO-d₆: δ 8.99 (s, 1H), 8.30-8.29 (m, 2H), 8.28-8.21 (m, 1H), 7.95-7.89 (m, 1H), 7.87-7.83 (m, 1H), 7.83-7.82 (m, 1H), 7.60-7.54 (m, 1H), 7.42-7.31 (m, 1H), 5.74-5.67 (m, 1H), 1.62 (d, J = 6.8 Hz, 6H). |
| I-050 | 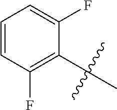 [SMILES: *c1c(F)cccc1F] | 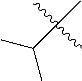 [SMILES: *C(C)C] | 368.0 | DMSO-d₆: δ 9.37 (s, 1H), 8.29-8.19 (m, 3H), 7.82-7.81 (m, 1H), 7.55-7.51 (m, 1H), 7.31-7.26 (m, 2H), 5.82-5.79 (m, 1H), 1.56 (d, J = 6.8 Hz, 6H). |
| I-051 | 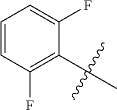 [SMILES: *c1c(F)cccc1F] | 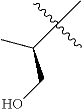 [SMILES: *[C@H](C)CO] | 384.1 | DMSO-d₆: δ 8.85 (s, 1H), 8.32-8.30 (m, 1H), 8.26-8.18 (m, 2H), 7.88 (s, 1H), 7.60-7.56 (m, 1H), 7.38-7.33 (m, 2H), 5.79-5.74 (m, 1H), 5.00-4.97 (m, 1H), 3.77-3.71 (m, 2H), 1.54 (d, J = 6.8 Hz, 3H). |
| I-052 | 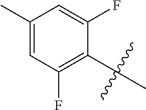 [SMILES: *c1c(F)cc(C)cc1F] | 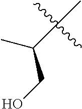 [SMILES: *[C@H](C)CO] | 398.1 | CD₃OD-d₄: δ 8.87 (s, 1H), 8.32-8.27 (m, 2H), 8.19-8.14 (m, 1H), 7.70-7.68 (m, 1H), 7.04 (d, J = 10.5 Hz, 2H), 6.03-5.92 (m, 1H), 4.00-3.87 (m, 2H), 2.43 (s, 3H), 1.66 (d, J = 6.9 Hz, 3H). |
| I-053 | 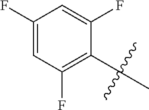 [SMILES: *c1c(F)cc(F)cc1F] | 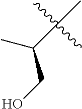 [SMILES: *[C@H](C)CO] | 402.0 | DMSO-d₆: δ 8.85 (s, 1H), 8.42-8.17 (m, 3H), 7.87 (s, 1H), 7.54-7.48 (m, 2H), 5.87-5.60 (m, 1H), 5.00-4.97 (m, 1H), 3.77-3.72 (m, 2H), 1.54 (d, J = 6.9 Hz, 3H). |
| I-054 | 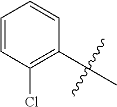 [SMILES: *c1ccccc1Cl] | 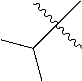 [SMILES: *C(C)C] | 366.1 | DMSO-d₆: δ 8.95 (s, 1H), 8.18-8.13 (m, 3H), 8.09 (s, 1H), 8.02-7.98 (m, 1H), 7.73-7.71 (m, 1H), 7.64-7.56 (m, 2H), 5.66-5.59 (m, 1H), 1.57-1.56 (d, J = 4.0 Hz, 6H). |
| I-055 | 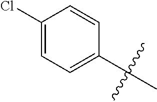 [SMILES: *c1ccc(Cl)cc1] | 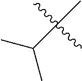 [SMILES: *C(C)C] | 366.1 | DMSO-d₆: δ 8.95 (s, 1H), 8.17-8.10 (m, 4H), 8.07-8.02 (m, 1H), 8.00 (s, 1H), 7.68 (d, J = 8.4 Hz, 2H), 5.60- 5.45 (m, 1H), 1.58 (d, J = 6.8 Hz, 6H). |
| I-056 | 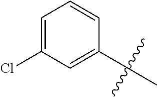 [SMILES: *c1cccc(Cl)c1] | 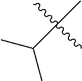 [SMILES: *C(C)C] | 366.1 | DMSO-d₆: δ 8.96 (s, 1H), 8.13-8.00 (m, 6H), 7.70-7.58 (m, 2H), 5.95-5.56 (m, 1H), 1.59 (d, J = 6.4 Hz, 6H). |
| I-057 | 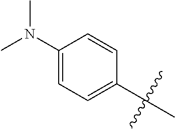 [SMILES: *c1ccc(N(C)C)cc1] | 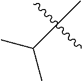 [SMILES: *C(C)C] | 375.2 | DMSO-d₆: δ 8.98 (s, 1H), 8.23-8.13 (m, 3H), 7.69-7.68 (m, 1H), 7.65-7.63 (m, 2H), 7.86-7.84 (m, 2H), 5.75-5.68 (m, 1H), 2.99 (s, 6H), 1.64 (d, J = 6.8 Hz, 6H). |
| I-058 | 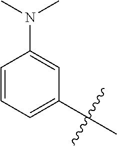 [SMILES: *c1cccc(N(C)C)c1] | 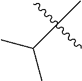 [SMILES: *C(C)C] | 375.3 | DMSO-d₆: δ 8.98 (s, 1H), 8.28-8.25 (m, 2H), 8.20-8.18 (m, 1H), 7.96 (s, 1H), 7.35-7.32 (m, 1H), 7.14-7.11 (m, 2H), 6.81-6.78 (m, 1H), 5.68-5.61 (m, 1H), 2.99 (s, 6H), 1.64 (d, J = 6.8 Hz, 6H). |
| I-059 | 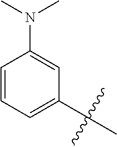 [SMILES: *c1cccc(N(C)C)c1] | 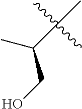 [SMILES: *[C@H](C)CO] | 391.2 | DMSO-d₆: δ 8.86 (s, 1H), 8.26-8.24 (m, 2H), 8.20-8.16 (m, 1H), 7.95 (s, 1H), 7.35-7.31 (m, 1H), 7.16-7.14 (m, 2H), 6.80-6.78 (m, 1H), 5.60-5.56 (m, 1H), 5.09-5.06 (m, 1H), 3.86-3.78 (m, 2H), 2.98 (s, 6H), 1.58 (d, J = 6.8 Hz, 3H). |
| I-060 | 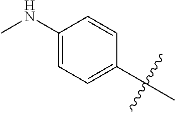 [SMILES: *c1ccc(NC)cc1] | 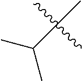 [SMILES: *C(C)C] | 361.2 | CD₃OD-d₄: δ 8.91 (s, 1H), 8.25-8.20 (m, 2H), 8.16-8.12 (m, 1H), 7.61-7.55 (m, 2H), 7.45 (s, 1H), 6.78-6.69 (m, 2H), 5.91-5.84 (m, 1H), 2.84 (s, 3H), 1.70 (d, J = 6.8 Hz, 6H). |
| I-061 | 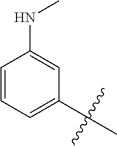 [SMILES: *c1cccc(NC)c1] | 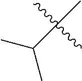 [SMILES: *C(C)C] | 361.0 | DMSO-d₆: δ 8.98 (s, 1H), 8.27-8.23 (m, 2H), 8.20-8.16 (m, 1H), 7.88 (s, 1H), 7.25-7.21 (m, 1H), 7.03 (d, J = 8.0 Hz, 1H), 6.92 (s, 1H), 6.64-6.61 (m, 1H), 5.93-5.90 (m, 1H), 5.68-5.61 (m, 1H), 2.76 (d, J = 5.2 Hz, 3H), 1.64 (d, J = 6.8 Hz, 6H). |
| I-062 | 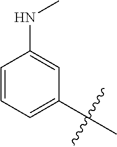 [SMILES: *c1cccc(NC)c1] | 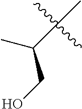 [SMILES: *[C@H](C)CO] | 377.2 | DMSO-d₆: δ 8.96 (s, 1H), 8.26-8.17 (m, 3H), 7.88 (s, 1H), 7.26-7.22 (m, 1H), 7.08 (d, J = 7.2 Hz, 1H), 6.97 (d, J = 7.2 Hz, 1H), 6.66 (d, J = 8.0 Hz, 1H), 5.64-5.57 (m, 1H), 3.89-3.78 (m, 2H), 2.76 (s, 3H), 1.60 (d, J = 7.2 Hz, 3H). |
| I-063 | 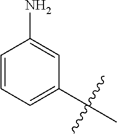 [SMILES: *c1cccc(N)c1] | 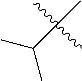 [SMILES: *C(C)C] | 347.2 | DMSO-d₆: δ 8.98 (s, 1H), 8.26-8.16 (m, 3H), 7.79 (s, 1H), 7.20-7.16 (m, 1H), 6.98 (d, J = 6.4 Hz, 2H), 6.63 (d, J = 8.4 Hz, 1H), 5.68-5.61 (m, 1H), 5.31 (s, 2H), 1.63 (d, J = 6.8 Hz, 6H). |
| I-064 | 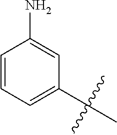 [SMILES: *c1cccc(N)c1] | 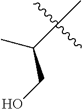 [SMILES: *[C@H](C)CO] | 363.1 | DMSO-d₆: δ 8.87 (s, 1H), 8.28-8.14 (m, 3H), 7.79 (s, 1H), 7.21-7.13 (m, 1H), 7.02-6.99 (m, 2H), 6.64-6.61 (m, 1H), 5.62-5.57 (m, 1H), 5.29 (s, 2H), 5.11-5.08 (m, 1H), 3.88-3.77 (m, 2H), 1.59 (d, J = 6.8 Hz, 3H). |
| I-065 | 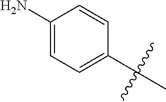 [SMILES: *c1ccc(N)cc1] | 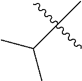 [SMILES: *C(C)C] | 347.2 | CD₃OD-d₄: δ 9.14 (s, 1H), 8.33-8.25 (m, 2H), 8.20-8.17 (m, 1H), 7.84-7.80 (m, 2H), 7.70 (s, 1H), 7.25-7.22 (m, 2H), 5.89-5.82 (m, 1H), 1.72 (d, J = 6.8 Hz, 6H). |
| I-066 | 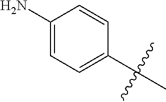 [SMILES: *c1ccc(N)cc1] | 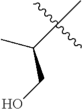 [SMILES: *[C@H](C)CO] | 363.3 | DMSO-d₆: δ 8.86 (s, 1H), 8.22-8.12 (m, 3H), 7.60 (s, 1H), 7.58-7.51 (m, 2H), 6.66 (d, J = 8.4 Hz, 2H), 5.65- 5.59 (m, 3H), 5.14 (s, 1H), 3.87-3.78 (m, 2H), 1.58 (d, J = 6.8 Hz, 3H). |
| I-067 | 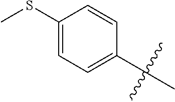 [SMILES: *c1ccc(SC)cc1] | 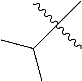 [SMILES: *C(C)C] | 378.2 | CD₃OD-d₄: δ 8.90 (s, 1H), 8.27-8.22 (m, 2H), 8.16-8.12 (m, 1H), 7.72-7.66 (m, 3H), 7.35 (d, J = 8.4 Hz, 2H), 5.85-5.78 (m, 1H), 2.54 (s, 3H), 1.68 (d, J = 6.8 Hz, 6H). |
| I-068 | 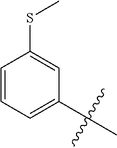 [SMILES: *c1cccc(SC)c1] | 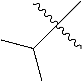 [SMILES: *C(C)C] | 378.0 | DMSO-d₆: δ 8.98 (s, 1H), 8.28-8.18 (m, 3H), 8.03 (s, 1H), 7.69 (s, 1H), 7.60 (d, J = 7.2 Hz, 1H), 7.50-7.46 (m, 1H), 7.31 (d, J = 7.6 Hz, 1H), 5.72- 5.69 (m, 1H), 2.56 (s, 3H), 1.65 (d, J = 6.4 Hz, 6H). |
| I-069 | 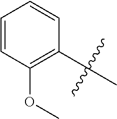 [SMILES: *c1ccccc1OC] | 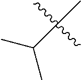 [SMILES: *C(C)C] | 362.0 | CD₃OD-d₄: δ 8.91 (s, 1H), 8.14-8.11 (m, 2H), 8.10-8.03 (m, 1H), 7.99-7.97 (m, 1H), 7.88 (s, 1H), 7.59-7.55 (m, 1H), 7.24-7.26 (d, J = 8 Hz, 1H), 7.24- 7.12 (m, 1H), 5.71-5.64 (m, 1H), 4.01 (s, 3H), 1.69-1.67 (d, J = 6.8 Hz, 6H). |
| I-070 | 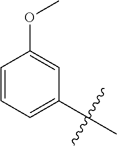 [SMILES: *c1cccc(OC)c1] | 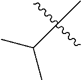 [SMILES: *C(C)C] | 362.2 | CD₃OD-d₄: δ 8.90 (s, 1H), 8.10-8.09 (m, 2H), 7.99-7.95 (m, 1H), 7.84-7.83 (m, 1H), 7.73-7.70 (m, 1H), 7.70-7.69 (m, 1H), 7.48-7.43 (m, 1H), 7.43-7.39 (m, 1H), 5.70-5.63 (m, 1H), 3.90-3.87 (m, 3H), 1.69-1.67 (d, J = 6.8 Hz, 6H). |
| I-071 | 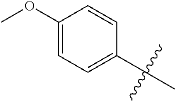 [SMILES: *c1ccc(OC)cc1] | 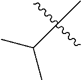 [SMILES: *C(C)C] | 362.2 | CD₃OD-d₄: δ 8.90 (s, 1H), 8.23-8.08 (m, 4H), 7.96-7.93 (m, 1H), 7.78 (s, 1H), 7.10-7.04 (m, 2H), 5.68-5.62 (m, 1H), 3.89-3.86 (m, 3H), 1.69-1.67 (d, J = 6.8 Hz, 6H). |
| I-072 | 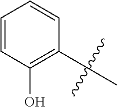 [SMILES: *c1ccccc1O] | 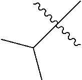 [SMILES: *C(C)C] | 348.1 | DMSO-d₆: δ 10.62 (s, 1H), 8.99 (s, 1H), 8.27-8.25 (m, 2H), 8.20-8.16 (m, 1H), 7.77-7.74 (m, 2H), 7.28-7.24 (m, 1H), 7.05-6.95 (m, 2H), 5.78-5.71 (m, 1H), 1.63 (d, J = 6.4 Hz, 6H). |
| I-073 | 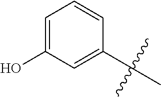 [SMILES: *c1cccc(O)c1] | 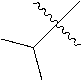 [SMILES: *C(C)C] | 348.2 | DMSO-d₆: δ 9.77 (s, 1H), 8.98 (s, 1H), 8.28-8.17 (m, 3H), 7.92 (s, 1H), 7.36- 7.32 (m, 1H), 7.28-7.26 (m, 1H), 7.21 (d, J = 1.6 Hz, 1H), 6.86-6.84 (m, 1H), 5.71-5.68 (m, 1H), 1.63 (d, J = 6.8 Hz, 6H). |
| I-074 | 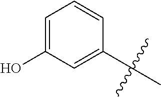 [SMILES: *c1cccc(O)c1] | 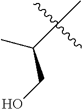 [SMILES: *[C@H](C)CO] | 364.2 | DMSO-d₆: δ 9.76 (s, 1H), 8.87 (s, 1H), 8.27-8.16 (m, 3H), 7.91 (s, 1H), 7.35- 7.28 (m, 2H), 7.21 (d, J = 2.0 Hz, 1H), 6.86-6.83 (m, 1H), 5.65-5.60 (m, 1H), 5.08-5.05 (m, 1H), 3.86-3.79 (m, 2H), 1.60 (d, J = 6.8 Hz, 3H) |
| I-075 | 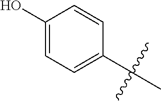 [SMILES: *c1ccc(O)cc1] | 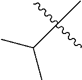 [SMILES: *C(C)C] | 348.2 | DMSO-d₆: δ 9.99 (s, 1H), 8.98 (s, 1H), 8.25-8.14 (m, 3H), 7.75 (s, 1H), 7.65 (d, J = 8.8 Hz, 2H), 6.92 (d, J = 8.4 Hz, 2H), 5.69-5.66 (m, 1H), 1.62 (d, J = 6.4 Hz, 6H). |
| I-076 | 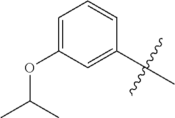 [SMILES: *c1cccc(OC(C)C)c1] | 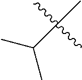 [SMILES: *C(C)C] | 390.2 | DMSO-d₆: δ 8.98 (s, 1H), 8.29-8.25 (m, 2H), 8.21-8.16 (m, 1H), 8.01 (s, 1H), 7.47-7.37 (m, 3H), 7.01-6.98 (m, 1H), 5.75-5.66 (m, 1H), 4.76-4.68 (m, 1H), 1.63 (d, J = 6.6 Hz, 6H), 1.31 (d, J = 6.0 Hz, 6H). |
| I-077 | 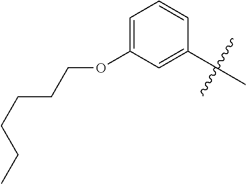 [SMILES: *c1cccc(OCCCCCC)c1] | 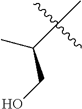 [SMILES: *[C@H](C)CO] | 448.1 | DMSO-d₆: δ 8.90 (s, 1H), 8.29-8.21 (m, 2H), 8.19-8.16 (m, 1H), 8.02 (s, 1H), 7.45-7.40 (m, 3H), 7.02-7.01 (m, 1H), 5.76-5.68 (m, 1H), 5.11-5.05 (m, 1H), 4.08-4.03 (m, 2H), 3.85-3.80 (m, 2H), 1.78-1.73 (m, 2H), 1.61 (d, J = 6.9 Hz, 3H), 1.48-1.43 (m, 2H), 1.36- 1.31 (m, 4H), 0.92-0.88 (m, 3H). |
| I-078 | 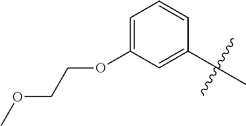 [SMILES: *c1cccc(OCCOC)c1] | 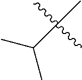 [SMILES: *C(C)C] | 406.2 | DMSO-d₆: δ 8.98 (s, 1H), 8.28-8.25 (m, 2H), 8.21-8.17 (m, 1H), 8.02 (s, 1H), 7.48-7.41 (m, 3H), 7.02 (d, J = 8.0 Hz, 1H), 5.72-5.68 (m, 1H), 4.20- 4.17 (m, 2H), 3.71-3.69 (m, 2H), 3.34 (s, 3H), 1.63 (d, J = 6.8 Hz, 6H). |
| I-079 | 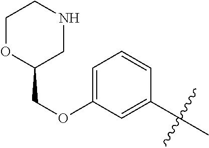 [SMILES: *c1cccc(OC[C@@H]2CNCCO2)c1] | 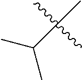 [SMILES: *C(C)C] | 447.2 | DMSO-d₆: δ 8.91 (s, 1H), 8.22-8.10 (m, 4H), 7.95 (s, 1H), 7.41-7.33 (m, 3H), 6.96-6.93 (m, 1H), 5.67-5.61 (m, 1H), 3.97-3.93 (m, 2H), 3.77-3.72 (m, 2H), 2.94-2.91 (m, 1H), 2.76-2.64 (m, 2H), 2.59-2.54 (m, 1H), 1.57 (m, J = 6.8 Hz, 6H). |
| I-080 | 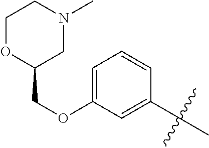 [SMILES: *c1cccc(OC[C@@H]2CN(C)CCO2)c1] | 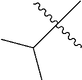 [SMILES: *C(C)C] | 461.3 | DMSO-d₆: δ 9.00 (s, 1H), 8.29-8.25 (m, 2H), 8.21-8.17 (m, 1H), 8.02 (s, 1H), 7.48-7.40 (m, 3H), 7.05-6.98 (m, 1H), 5.74-5.71 (m, 1H), 4.07-4.04 (m, 2H), 3.84-3.80 (m, 2H), 3.59-3.54 (m, 1H), 2.88-2.80 (m, 1H), 2.65-2.60 (m, 1H), 2.22 (s, 3H), 2.05-2.02 (m, 1H), 1.95-1.89 (m, 1H), 1.64 (m, J = 6.8 Hz, 6H). |
| I-081 | 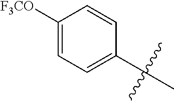 [SMILES: *c1ccc(OC(F)(F)F)cc1] | 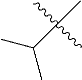 [SMILES: *C(C)C] | 416.2 | CD₃OD-d₄: δ 8.92 (s, 1H), 8.34-8.17 (m, 3H), 7.95-7.94 (m, 2H), 7.81 (s, 1H), 7.45-7.43 (m, 2H), 5.85-5.79 (m, 1H), 1.70 (d, J = 6.8 Hz, 6H). |
| I-082 | 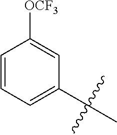 [SMILES: *c1cccc(OC(F)(F)F)c1] | 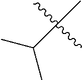 [SMILES: *C(C)C] | 416.0 | DMSO-d₆: δ 8.98 (s, 1H), 8.30-8.27 (m, 2H), 8.22-8.18 (m, 1H), 8.14 (s, 1H), 7.88 (d, J = 8.0 Hz, 1H), 7.79 (s, 1H), 7.72-7.68 (m, 1H), 7.46 (d, J = 8.4 Hz, 1H), 5.73-5.66 (m, 1H), 1.63 (d, J = 6.8 Hz, 6H). |
| I-083 | 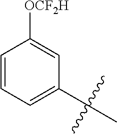 | 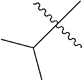 [SMILES: *C(C)C] | 398.2 | DMSO-d₆: δ 8.99 (s, 1H), 8.30-8.27 (m, 2H), 8.22-8.18 (m, 1H), 8.09 (s, 1H), 7.73 (d, J = 8.0 Hz, 1H), 7.64- 7.54 (m, 2H), 7.35-7.17 (m, 2H), 5.74- 5.67 (m, 1H), 1.64 (d, J = 6.8 Hz, 6H). |
| I-084 | 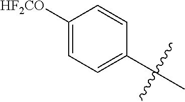 | 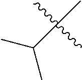 [SMILES: *C(C)C] | 398.3 | CD₃OD-d₄: δ 8.92 (s, 1H), 8.28-8.24 (m, 2H), 8.17-8.15 (m, 1H), 7.86 (s, 2H), 7.75-7.72 (m, 1H), 7.29 (s, 2H), 5.81 (s, 1H), 1.71 (d, J = 6.4 Hz, 6H). |
| I-085 | 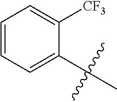 [SMILES: *c1ccccc1C(F)(F)F] | 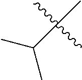 [SMILES: *C(C)C] | 400.0 | DMSO-d₆: δ 8.96 (s, 1H), 8.33-8.31 (m, 1H), 8.28-8.19 (m, 2H), 7.98-7.96 (m, 2H), 7.89-7.83 (m, 2H), 7.75-7.71 (m, 1H), 5.81-5.74 (m, 1H), 1.51 (d, J = 6.4 Hz, 6H). |
| I-086 | 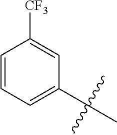 [SMILES: *c1cccc(C(F)(F)F)c1] | 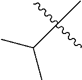 [SMILES: *C(C)C] | 400.0 | DMSO-d₆: δ 8.99 (s, 1H), 8.31-8.28 (m, 2H), 8.22-8.13 (s, 4H), 7.81 (d, J = 4.4 Hz, 2H), 5.76-5.69 (m, 1H), 1.64 (d, J = 6.4 Hz, 6H). |
| I-087 | 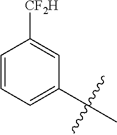 [SMILES: *c1cccc(C(F)F)c1] | 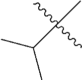 [SMILES: *C(C)C] | 382.0 | DMSO-d₆: δ 8.99 (s, 1H), 8.31-8.27 (m, 2H), 8.22-8.18 (m, 1H), 8.12 (s, 1H), 8.04-8.02 (m, 2H), 7.73-7.69 (m, 1H), 7.63 (d, J = 8.0 Hz, 1H), 7.29- 7.01 (m, 1H), 5.79-5.72 (m, 1H), 1.64 (d, J = 6.4 Hz, 6H). |
| I-088 | 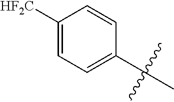 | 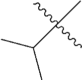 [SMILES: *C(C)C] | 382.2 | CD₃OD-d₄: δ 8.89 (s, 1H), 8.27-8.25 (m, 2H), 8.14-8.10 (m, 2H), 8.04-8.01 (m, 1H), 7.89 (s, 1H), 7.74 (d, J = 8.0 Hz, 2H), 7.01-6.73 (m, 1H), 5.69-5.62 (m, 1H), 1.67 (d, J = 6.4 Hz, 6H). |
| I-089 | 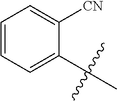 [SMILES: *c1ccccc1C#N] | 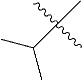 [SMILES: *C(C)C] | 357.1 | DMSO-d₆: δ 8.96 (s, 1H), 8.32-8.29 (m, 2H), 8.24-8.20 (m, 1H), 8.12 (d, J = 1.6 Hz, 1H), 8.06-8.01 (m, 2H), 7.93-7.89 (m, 1H), 7.67-7.63 (m, 1H), 5.76-5.69 (m, 1H), 1.41 (d, J = 7.2 Hz, 6H). |
| I-090 | 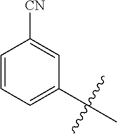 [SMILES: *c1cccc(C#N)c1] | 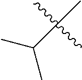 [SMILES: *C(C)C] | 357.0 | DMSO-d₆: δ 8.98 (s, 1H), 8.31-8.28 (m, 3H), 8.23-8.19 (s, 1H), 8.15 (d, J = 6.4 Hz, 2H), 7.88 (d, J = 7.6 Hz, 1H), 7.79-7.51 (m, 1H), 5.71-5.68 (m, 1H), 1.64 (d, J = 6.8 Hz, 6H). |
| I-091 | 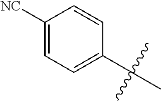 [SMILES: *c1ccc(C#N)cc1] | 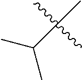 [SMILES: *C(C)C] | 357.1 | CD₃OD-d₄: δ 8.92 (s, 1H), 8.36-8.34 (m, 1H), 8.30-8.28 (m, 1H), 8.21-8.17 (m, 1H), 8.01-7.96 (m, 2H), 7.91 (s, 1H), 7.88 (d, J = 8.4 Hz, 2H), 5.84- 5.78 (m, 1H), 1.71 (d, J = 6.8 Hz, 6H). |
| I-092 | 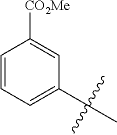 [SMILES: *c1cccc(C(=O)OC)c1] | 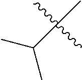 [SMILES: *C(C)C] | 390.0 | DMSO-d₆: δ 8.99 (s, 1H), 8.39 (s, 1H), 8.32-8.26 (m, 2H), 8.22-8.18 (m, 1H), 8.16-8.13 (m, 2H), 8.13-8.00 (m, 1H), 7.73-7.69 (m, 1H), 5.83-5.77 (m, 1H), 3.93 (s, 3H), 1.68 (d, J = 6.8 Hz, 6H). |
| I-093 | 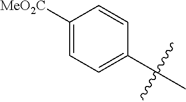 [SMILES: *c1ccc(C(=O)OC)cc1] | 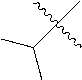 [SMILES: *C(C)C] | 390.2 | CD₃OD-d₄: δ 8.91 (s, 1H), 8.32-8.25 (m, 2H), 8.18-8.07 (m, 3H), 7.92-7.88 (m, 3H), 5.85-5.78 (m, 1H), 3.93 (s, 3H), 1.70 (d, J = 6.8 Hz, 6H). |
| I-094 | 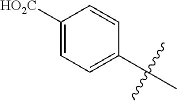 [SMILES: *c1ccc(C(=O)O)cc1] | 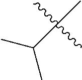 [SMILES: *C(C)C] | 376.2 | CD₃OD-d₄: δ 8.93 (s, 1H), 8.36-8.34 (m, 1H), 8.30-8.28 (m, 1H), 8.22-8.15 (m, 3H), 7.96-7.91 (m, 3H), 5.89-5.82 (m, 1H), 1.72 (d, J = 6.8 Hz, 6H). |
| I-095 | 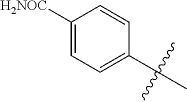 [SMILES: *c1ccc(C(N)=O)cc1] | 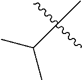 [SMILES: *C(C)C] | 375.2 | CD₃OD-d₄: δ 8.93 (s, 1H), 8.35-8.33 (m, 1H), 8.30-8.27 (m, 1H), 8.21-8.17 (m, 1H), 8.04-8.02 (m, 2H), 8.96-7.93 (m, 2H), 7.89 (m, 1H), 5.89-5.83 (m, 1H), 1.72 (d, J = 6.4 Hz, 6H). |
| I-096 | 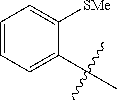 [SMILES: *c1ccccc1SC] | 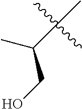 [SMILES: *[C@H](C)CO] | 394.1 | DMSO-d₆: δ 8.85 (s, 1H), 8.29-8.25 (m, 2H), 8.21-8.17 (m, 1H), 7.88-7.84 (m, 2H), 7.49-7.44 (m, 2H), 7.36-7.32 (m, 1H), 5.73-5.68 (m, 1H), 5.07-5.04 (m, 1H), 3.82-3.75 (m, 2H), 2.58 (s, 3H), 1.56 (d, J = 6.8 Hz, 3H). |
| I-097 | 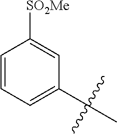 [SMILES: *c1cccc(S(C)(=O)=O)c1] | 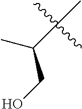 [SMILES: *[C@H](C)CO] | 426.1 | DMSO-d₆: δ 8.89 (s, 1H), 8.38-8.26 (m, 3H), 8.24-8.17 (m, 3H), 7.99-7.97 (m, 1H), 7.86-7.82 (m, 1H), 5.74-5.70 (m, 1H), 5.07-5.04 (m, 1H), 3.86-3.83 (m, 2H), 3.32 (s, 3H), 1.64 (d, J = 6.8 Hz, 3H). |
| I-098 | 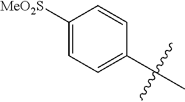 [SMILES: *c1ccc(S(C)(=O)=O)cc1] | 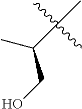 [SMILES: *[C@H](C)CO] | 426.0 | DMSO-d₆: δ 8.89 (s, 1H), 8.31-8.28 (m, 2H), 8.23-8.19 (m, 2H), 8.14-8.08 (m, 4H), 5.69-5.65 (m, 1H), 5.15-5.12 (m, 1H), 3.89-3.82 (m, 2H), 3.29 (s, 3H), 1.61 (d, J = 7.2 Hz, 3H). |
| I-099 | 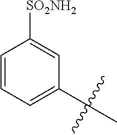 [SMILES: *c1cccc(S(N)(=O)=O)c1] | 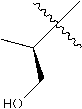 [SMILES: *[C@H](C)CO] | 427.1 | DMSO-d₆: δ 8.89 (s, 1H), 8.34-8.25 (m, 3H), 8.22-8.19 (m, 1H), 8.16-8.07 (m, 2H), 7.88-7.86 (m, 1H), 7.78-7.74 (m, 1H), 7.45 (s, 2H), 5.71-5.67 (m, 1H), 5.08 (s, 1H), 3.85-3.83 (m, 2H), 1.64 (d, J = 6.8 Hz, 3H). |
| I-100 | 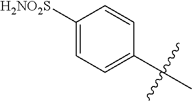 [SMILES: *c1ccc(S(N)(=O)=O)cc1] | 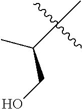 [SMILES: *[C@H](C)CO] | 426.8 | DMSO-d₆: δ 8.89 (s, 1H), 8.32-8.27 (m, 2H), 8.23-8.18 (m, 1H), 8.14 (s, 1H), 8.06 (d, J = 8.4 Hz, 2H), 7.98 (d, J = 8.4 Hz, 2H), 7.46 (s, 2H), 5.66- 5.62 (m, 1H), 5.16-5.12 (m, 1H), 3.90- 3.80 (m, 2H), 1.60 (d, J = 6.9 Hz, 3H). |
| I-101 | 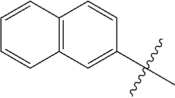 [SMILES: *c1ccc2ccccc2c1] | 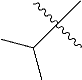 [SMILES: *C(C)C] | 382.2 | DMSO-d₆: δ 8.98 (s, 1H), 8.76-8.75 (m, 1H), 8.23-8.13 (m, 6H), 8.12-8.03 (m, 2H), 7.67-7.65 (m, 2H), 5.62-5.58 (m, 1H), 1.63 (d, J = 6.8 Hz, 6H). |
| I-102 | 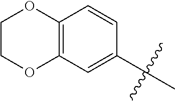 [SMILES: *c1ccc2c(c1)OCCO2] | 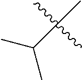 [SMILES: *C(C)C] | 390.1 | DMSO-d₆: δ 8.99 (s, 1H), 8.26-8.15 (m, 3H), 7.85 (s, 1H), 7.34-7.30 (m, 2H), 7.03 (d, J = 8.4 Hz, 1H), 5.74-5.67 (m, 1H), 4.31 (s, 4H), 1.62 (d, J = 6.8 Hz, 6H). |
| I-103 | 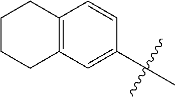 [SMILES: *c1ccc2c(c1)CCCC2] | 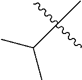 [SMILES: *C(C)C] | 386.2 | DMSO-d₆: δ 8.98 (s, 1H), 8.27-8.15 (m, 3H), 7.88 (s, 1H), 7.55-7.53 (m, 2H), 7.22-7.20 (m, 1H), 5.77-5.70 (m, 1H), 2.79-2.76 (m, 4H), 1.85-1.77 (m, 4H), 1.63 (d, J = 6.8 Hz, 6H). |
| I-104 | 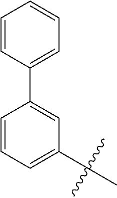 [SMILES: *c1cccc(-c2ccccc2)c1] | 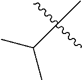 [SMILES: *C(C)C] | 408.3 | DMSO-d₆: δ 8.97 (s, 1H), 8.29-8.26 (m, 2H), 8.20-8.17 (m, 1H), 8.09 (d, J = 2.8 Hz, 2H), 7.84 (d, J = 8.0 Hz, 1H), 7.76-7.70 (m, 3H), 7.65-7.61 (m, 1H), 7.54-7.50 (m, 2H), 7.46-7.42 (m, 1H), 5.74-5.68 (m, 1H), 1.61 (d, J = 6.8 Hz, 6H). |
| I-105 | 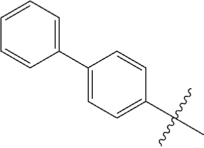 [SMILES: *c1ccc(-c2ccccc2)cc1] | 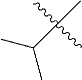 [SMILES: *C(C)C] | 408.2 | CD₃OD-d₄: δ 8.90 (s, 1H), 8.30-8.24 (m, 2H), 8.17-8.13 (m, 1H), 7.89-7.87 (m, 2H), 7.77-7.75 (m, 3H), 7.68-7.66 (m, 2H), 7.48-7.44 (m, 3H), 5.87-5.81 (m, 1H), 1.70 (d, J = 6.4 Hz, 6H). |
| I-106 | 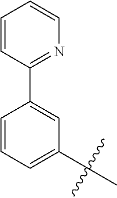 [SMILES: *c1cccc(-c2ccccn2)c1] | 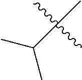 [SMILES: *C(C)C] | 409.2 | DMSO-d₆: δ 8.99 (s, 1H), 8.72 (d, J = 4.4 Hz, 1H), 8.61 (s, 1H), 8.32-8.27 (m, 2H), 8.22-8.18 (m, 1H), 8.13-8.07 (m, 3H), 7.97-7.92 (m, 2H), 7.69-7.65 (m, 1H), 7.46-7.43 (m, 1H), 5.86-5.79 (m, 1H), 1.69 (d, J = 6.8 Hz, 6H). |
| I-107 | 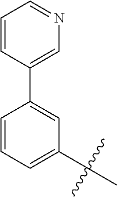 [SMILES: *c1cccc(-c2cccnc2)c1] | 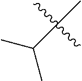 [SMILES: *C(C)C] | 409.2 | DMSO-d₆: δ 9.00-8.98 (m, 2H), 8.66- 8.64 (m, 1H), 8.30-8.27 (m, 2H), 8.22- 8.17 (m, 3H), 8.14 (s, 1H), 7.89 (d, J = 8.0 Hz, 1H), 7.79 (d, J = 8.0 Hz, 1H), 7.71-7.67 (m, 1H), 7.56-7.53 (m, 1H), 5.74-5.67 (m, 1H), 1.62 (d, J = 6.8 Hz, 6H). |
| I-108 | 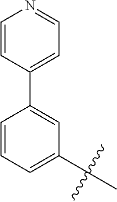 [SMILES: *c1cccc(-c2ccncc2)c1] | 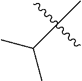 [SMILES: *C(C)C] | 409.2 | DMSO-d₆: δ 8.98 (s, 1H), 8.71-8.70 (m, 2H), 8.30-8.27 (m, 2H), 8.22-8.19 (m, 2H), 8.14 (s, 1H), 7.93 (d, J = 8.0 Hz, 1H), 7.85 (d, J = 8.0 Hz, 1H), 7.81-7.80 (m, 2H), 7.73-7.69 (m, 1H), 5.72-5.66 (m, 1H), 1.61 (d, J = 6.8 Hz, 6H). |
| I-109 | 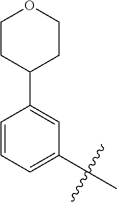 [SMILES: *c1cccc(C2CCOCC2)c1] | 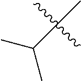 [SMILES: *C(C)C] | 416.1 | DMSO-d₆: δ 8.99 (s, 1H), 8.28-8.26 (m, 2H), 8.21-8.17 (m, 1H), 8.01 (s, 1H), 7.73-7.72 (m, 1H), 7.70-7.68 (m, 1H), 7.50-7.46 (m, 1H), 7.35-7.33 (m, 1H), 5.74-5.67 (m, 1H), 4.00-3.97 (m, 2H), 3.50-3.43 (m, 2H), 2.89-2.81 (m, 1H), 1.77-1.69 (m, 4H), 1.64 (d, J = 6.8 Hz, 6H). |
| I-110 | 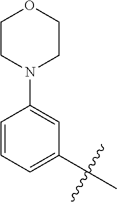 [SMILES: *c1cccc(N2CCOCC2)c1] | 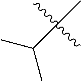 [SMILES: *C(C)C] | 417.1 | DMSO-d₆: δ 8.97 (s, 1H), 8.27-8.25 (m, 2H), 8.21-8.17 (m, 1H), 7.98 (s, 1H), 7.41-7.37 (m, 2H), 7.29 (d, J = 7.6 Hz, 1H), 7.04-7.01 (m, 1H), 5.59- 5.62 (m, 1H), 3.79-3.77 (m, 4H), 3.22- 3.19 (m, 4H), 1.63 (d, J = 6.8 Hz, 6H). |
| I-111 | 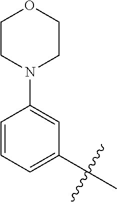 [SMILES: *c1cccc(N2CCOCC2)c1] | 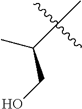 [SMILES: *[C@H](C)CO] | 433.2 | DMSO-d₆: δ 8.67 (s, 1H), 8.27-8.24 (m, 2H), 8.20-8.16 (m, 1H), 7.98 (s, 1H), 7.40-7.36 (m, 2H), 7.32-7.29 (m, 1H), 7.03-7.00 (m, 1H), 5.61-5.56 (m, 1H), 5.12-5.09 (m, 1H), 3.87-3.76 (m, 6H), 3.22-3.19 (m, 4H), 1.59 (d, J = 7.2 Hz, 3H). |
| I-112 | 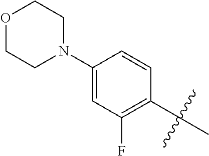 [SMILES: *c1ccc(N2CCOCC2)cc1F] | 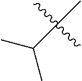 [SMILES: *C(C)C] | 435.3 | DMSO-d₆: δ 8.95 (s, 1H), 8.11 (s, 2H), 7.97-7.93 (m, 3H), 6.97-6.93 (m, 2H), 5.66-5.63 (m, 1H), 3.75 (s, 4H), 3.32 (s, 4H), 1.58 (d, J = 6.4 Hz, 6H). |
| I-113 | 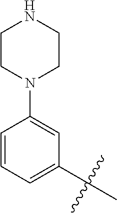 [SMILES: *c1cccc(N2CCNCC2)c1] | 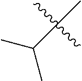 [SMILES: *C(C)C] | 416.1 | DMSO-d₆: δ 8.98 (s, 1H), 8.28-8.26 (m, 2H), 8.21-8.17 (m, 1H), 7.98 (s, 1H), 7.39-7.34 (m, 2H), 7.25 (d, J = 7.6 Hz, 1H), 7.00 (d, J = 8.0 Hz, 1H), 5.71-5.64 (m, 1H), 3.15-3.12 (m, 4H), 2.88-2.86 (m, 4H), 1.64 (d, J = 6.8 Hz, 6H). |
| I-114 | 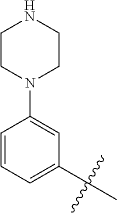 [SMILES: *c1cccc(N2CCNCC2)c1] | 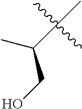 [SMILES: *[C@H](C)CO] | 432.3 | DMSO-d₆: δ 8.88 (s, 1H), 8.27-8.25 (m, 3H), 8.20-8.16 (m, 1H), 7.97 (s, 1H), 7.39-7.35 (m, 2H), 7.30-7.27 (m, 1H), 7.02-6.99 (m, 1H), 5.62-5.55 (m, 1H), 3.87-3.78 (m, 2H), 3.23-3.17 (m, 4H), 2.98-2.92 (m, 4H), 1.59 (d, J = 6.8 Hz, 3H). |
| I-115 | 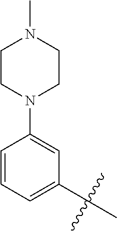 [SMILES: *c1cccc(N2CCN(C)CC2)c1] | 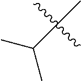 [SMILES: *C(C)C] | 430.3 | DMSO-d₆: δ 8.97 (s, 1H), 8.27-8.24 (m, 2H), 8.20-8.16 (m, 1H), 7.97 (s, 1H), 7.38-7.34 (m, 2H), 7.24 (d, J = 8.0 Hz, 1H), 7.01-6.99 (m, 1H), 5.70- 5.63 (m, 1H), 3.23-3.21 (m, 4H), 2.51- 2.47 (m, 4H), 2.24 (s, 3H), 1.62 (d, J = 6.4 Hz, 6H). |
| I-116 | 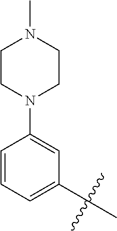 [SMILES: *c1cccc(N2CCN(C)CC2)c1] | 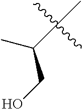 [SMILES: *[C@H](C)CO] | 446.4 | DMSO-d₆: δ 8.87 (s, 1H), 8.30-8.23 (m, 2H), 8.20-8.15 (m, 1H), 7.98 (s, 1H), 7.41-7.32 (m, 2H), 7.28-7.26 (s, 1H), 7.02-6.99 (m, 1H), 5.62-5.58 (m, 1H), 5.11-5.08 (m, 1H), 3.88-3.77 (m, 2H), 3.24-3.21 (m, 4H), 2.50-2.45 (m, 4H), 2.25 (s, 3H), 1.60 (d, J = 6.8 Hz, 3H). |
| I-117 | 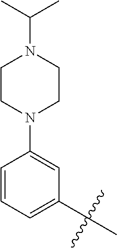 [SMILES: *c1cccc(N2CCN(C(C)C)CC2)c1] | 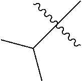 [SMILES: *C(C)C] | 458.3 | CD₃OD-d₄: δ 8.92 (s, 1H), 8.33-8.30 (m, 1H), 8.24-8.15 (m, 2H), 7.78 (s, 1H), 7.46-7.40 (m, 3H), 7.11-7.09 (m, 1H), 5.77-5.70 (m, 1H), 4.13-3.31 (m, 8H), 3.20-3.00 (m, 1H), 1.69 (d, J = 6.8 Hz, 6H), 1.43 (d, J = 6.4 Hz, 6H). |
| I-118 | 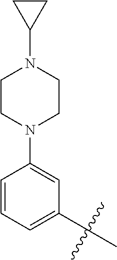 [SMILES: *c1cccc(N2CCN(C3CC3)CC2)c1] | 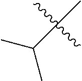 [SMILES: *C(C)C] | 456.4 | CD₃OD-d₄: δ 8.91 (s, 1H), 8.29-8.23 (m, 2H), 8.16-8.13 (m, 1H), 7.71 (s, 1H), 7.37-7.26 (m, 3H), 7.02-7.00 (m, 1H), 5.82-5.80 (m, 1H), 3.33-3.23 (m, 4H), 2.85-2.79 (m, 4H), 1.77-1.70 (m, 7H), 0.58-0.47 (m, 4H). |
| I-119 | 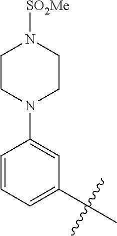 [SMILES: *c1cccc(N2CCN(S(C)(=O)=O)CC2)c1] | 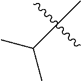 [SMILES: *C(C)C] | 494.2 | DMSO-d₆: δ 8.98 (s, 1H), 8.28-8.24 (m, 2H), 8.20-8.15 (m, 1H), 7.99 (s, 1H), 7.43-7.38 (m, 2H), 7.31-7.28 (m, 1H), 7.07-7.04 (m, 1H), 5.68-5.59 (m, 1H), 3.36-3.24 (m, 8H), 2.91 (s, 3H), 1.63 (d, J = 6.9 Hz, 6H). |
| I-120 | 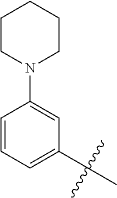 [SMILES: *c1cccc(N2CCCCC2)c1] | 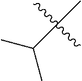 [SMILES: *C(C)C] | 415.3 | DMSO-d₆: δ 8.98 (s, 1H), 8.28-8.25 (m, 2H), 8.20-8.16 (m, 1H), 7.97 (s, 1H), 7.37-7.33 (m, 2H), 7.22 (d, J = 7.6 Hz, 1H), 7.00-6.98 (m, 1H), 5.71- 5.64 (m, 1H), 3.24-3.21 (m, 4H), 1.66- 1.57 (m, 12H). |
| I-121 | 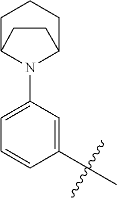 [SMILES: *c1cccc(N2C3CCCC2CC3)c1] | 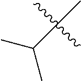 [SMILES: *C(C)C] | 441.3 | DMSO-d₆: δ 8.97 (s, 1H), 8.28-8.24 (m, 2H), 8.21-8.16 (m, 1H), 7.95 (s, 1H), 7.35-7.30 (m, 1H), 7.20-7.15 (m, 1H), 7.07 (d, J = 7.5 Hz, 1H), 6.85- 6.82 (m, 1H), 5.68-5.59 (m, 1H), 4.33- 4.27 (m, 2H), 2.08-2.00 (m, 2H), 1.93- 1.75 (m, 5H), 1.62 (d, J = 6.6 Hz, 6H), 1.45-1.42 (m, 1H), 1.31-1.28 (m, 2H). |
| I-122 | 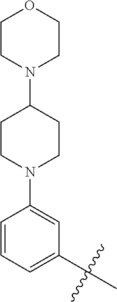 [SMILES: *c1cccc(N2CCC(N3CCOCC3)CC2)c1] | 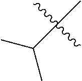 [SMILES: *C(C)C] | 500.3 | CD₃OD-d₄: δ 8.91 (s, 1H), 8.30-8.27 (m, 1H), 8.24-8.12 (m, 2H), 7.73 (s, 1H), 7.41-7.29 (m, 3H), 7.07-7.04 (m, 1H), 5.80-5.71 (m, 1H), 4.20-3.70 (m, 6H), 3.65-3.34 (m, 4H), 3.30-3.20 (m, 1H), 2.92-2.78 (m, 2H), 2.30-2.20 (m, 2H), 1.95-1.78 (m, 2H), 1.69 (d, J = 6.6 Hz, 6H). |
| I-123 | 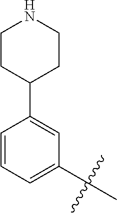 [SMILES: *c1cccc(C2CCNCC2)c1] | 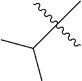 [SMILES: *C(C)C] | 415.1 | DMSO-d₆: δ 8.99 (s, 1H), 8.28-8.25 (m, 2H), 8.21-8.17 (m, 1H), 7.99 (s, 1H), 7.70-7.66 (m, 2H), 7.49-7.45 (m, 1H), 7.30 (d, J = 7.6 Hz, 1H), 5.75- 5.68 (m, 1H), 3.07-3.04 (m, 2H), 2.68- 2.58 (m, 3H), 1.76-1.70 (m, 2H), 1.65 (d, J = 6.8 Hz, 6H), 1.64-1.53 (m, 2H). |
| I-124 | 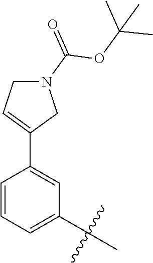 [SMILES: *c1cccc(C2=CCN(C(=O)OC(C)(C)C)C2)c1] | 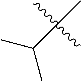 [SMILES: *C(C)C] | 499.2 | DMSO-d₆: δ 8.99 (s, 1H), 8.30-8.26 (m, 2H), 8.23-8.17 (m, 1H), 8.10-8.08 (m, 1H), 7.91-7.87 (m, 1H), 7.80-7.77 (m, 1H), 7.58-7.56 (m, 2H), 6.53-6.50 (m, 1H), 5.66-5.59 (m, 1H), 4.50-4.45 (m, 2H), 4.30-4.25 (m, 2H), 1.65 (d, J = 6.6 Hz, 6H), 1.47 (s, 9H). |
| I-125 | 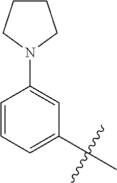 [SMILES: *c1cccc(N2CCCC2)c1] | 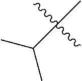 [SMILES: *C(C)C] | 401.2 | DMSO-d₆: δ 8.97 (s, 1H), 8.27-8.24 (m, 2H), 8.20-8.16 (m, 1H), 7.93 (s, 1H), 7.32-7.28 (m, 1H), 7.07 (d, J = 8.0 Hz, 1H), 6.93 (s, 1H), 6.12-6.59 (m, 1H), 5.68-5.61 (m, 1H), 3.32-3.28 (m, 4H), 2.01-1.98 (m, 4H), 1.64 (d, J = 6.8 Hz, 6H). |
| I-126 | 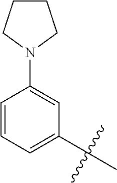 [SMILES: *c1cccc(N2CCCC2)c1] | 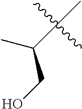 [SMILES: *[C@H](C)CO] | 417.2 | DMSO-d₆: δ 8.86 (s, 1H), 8.26-8.24 (m, 2H), 8.20-8.16 (m, 1H), 7.93 (s, 1H), 7.32-7.28 (m, 1H), 7.09 (d, J = 7.6 Hz, 1H), 6.97-6.95 (m, 1H), 6.62- 6.59 (m, 1H), 5.58-5.56 (m, 1H), 5.09- 5.06 (m, 1H), 3.90-3.70 (m, 2H), 3.33- 3.28 (m, 4H), 2.01-1.97 (m, 4H), 1.60 (d, J = 6.8 Hz, 3H). |
| I-127 | 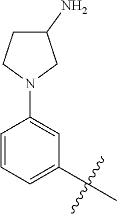 [SMILES: *c1cccc(N2CCC(N)C2)c1] | 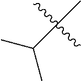 [SMILES: *C(C)C] | 416.2 | DMSO-d₆: δ 8.98 (s, 1H), 8.26-8.24 (m, 2H), 8.20-8.16 (m, 1H), 7.94 (s, 1H), 7.32-7.28 (m, 1H), 7.07-7.05 (m, 1H), 6.89 (s, 1H), 6.57-6.54 (m, 1H), 5.65-5.62 (m, 1H), 3.61-3.59 (m, 1H), 3.48-3.39 (m, 2H), 3.30-3.20 (m, 1H), 2.96-2.93 (m, 1H), 2.10-2.07 (m, 1H), 1.90-1.80 (m, 2H), 1.76-1.72 (m, 1H), 1.64-1.62 (m, 6H). |
| I-128 | 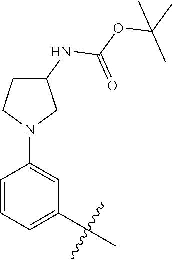 [SMILES: *c1cccc(N2CCC(NC(=O)OC(C)(C)C)C2)c1] | 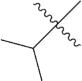 [SMILES: *C(C)C] | 516.4 | DMSO-d₆: δ 8.97 (s, 1H), 8.27-8.25 (m, 2H), 8.20-8.16 (m, 1H), 7.96 (s, 1H), 7.33-7.31 (m, 1H), 7.29-7.23 (m, 1H), 7.09 (d, J = 8.0 Hz, 1H), 6.90 (s, 1H), 6.59-6.56 (m, 1H), 5.66-5.62 (m, 1H), 4.18-4.10 (m, 1H), 3.55-3.51 (m, 1H), 3.44-3.40 (m, 1H), 3.32-3.29 (m, 1H), 3.13-3.09 (m, 1H), 2.20-2.15 (m, 1H), 1.96-1.91 (m, 1H), 1.63 (d, J = 6.8 Hz, 6H), 1.41 (s, 9H). |
| I-129 | 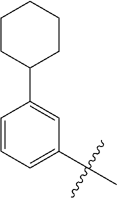 [SMILES: *c1cccc(C2CCCCC2)c1] | 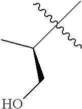 [SMILES: *[C@H](C)CO] | 430.2 | DMSO-d₆: δ 8.89 (s, 1H), 8.28-8.26 (m, 2H), 8.26-8.25 (m, 1H), 7.99 (s, 1H), 7.71-7.67 (m, 2H), 7.46-7.42 (m, 1H), 7.29 (d, J = 7.6 Hz, 1H), 5.70- 5.66 (m, 1H), 5.09-5.06 (m, 1H), 3.88- 3.82 (m, 2H), 2.60-2.53 (m, 1H), 1.88- 1.83 (m, 4H), 1.75-1.72 (m, 1H), 1.62 (d, J = 6.8 Hz, 3H), 1.54-1.26 (m, 5H). |
| I-130 | 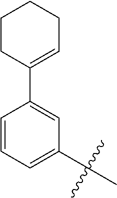 [SMILES: *c1cccc(C2=CCCCC2)c1] | 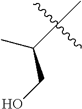 [SMILES: *[C@H](C)CO] | 428.1 | DMSO-d₆: δ 8.87 (s, 1H), 8.28-8.26 (m, 2H), 8.21-8.17 (m, 1H), 8.03 (s, 1H), 7.86 (s, 1H), 7.73 (d, J = 6.8 Hz, 1H), 7.48-7.46 (m, 2H), 6.28-6.27 (m, 1H), 5.65-5.56 (m, 1H), 5.09-5.06 (m, 1H), 3.84-3.80 (m, 2H), 2.49-2.43 (m, 2H), 2.24-2.21 (m, 2H), 1.78-1.75 (m, 2H), 1.66-1.62 (m, 5H). |
| I-131 | 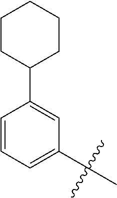 [SMILES: *c1cccc(C2CCCCC2)c1] | 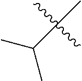 [SMILES: *C(C)C] | 414.2 | DMSO-d₆: δ 8.98 (s, 1H), 8.29-8.16 (m, 3H), 7.98 (s, 1H), 7.69-7.65 (m, 2H), 7.47-7.42 (m, 1H), 7.29 (d, J = 7.5 Hz, 1H), 5.77-5.68 (m, 1H), 2.62- 2.58 (m, 1H), 1.85-1.64 (m, 11H), 1.53-1.23 (m, 5H). |
| I-132 | 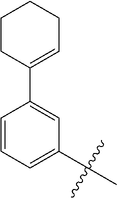 [SMILES: *c1cccc(C2=CCCCC2)c1] | 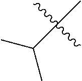 [SMILES: *C(C)C] | 412.2 | DMSO-d₆: δ 8.99 (s, 1H), 8.29-8.26 (m, 2H), 8.21-8.17 (m, 1H), 8.03 (s, 1H), 7.83 (s, 1H), 7.70 (d, J = 7.2 Hz, 1H), 7.51-7.44 (m, 2H), 6.58-6.53 (m, 1H), 5.75-5.68 (m, 1H), 2.43-2.33 (m, 2H), 2.22-2.15 (m, 2H), 1.88-1.73 (m, 2H), 1.67-1.62 (m, 8H). |
| I-133 | 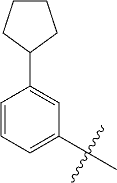 [SMILES: *c1cccc(C2CCCC2)c1] | 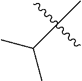 [SMILES: *C(C)C] | 400.2 | DMSO-d₆: δ 8.99 (s, 1H), 8.28-8.25 (m, 2H), 8.21-8.17 (m, 1H), 7.99 (s, 1H), 7.71 (s, 1H), 7.67-7.65 (m, 1H), 7.47-7.43 (m, 1H), 7.32 (d, J = 8.0 Hz, 1H), 5.72-5.65 (m, 1H), 3.06-3.02 (m, 1H), 2.10-2.06 (m, 2H), 1.83-1.78 (m, 2H), 1.74-1.57 (m, 10H). |
| I-134 | 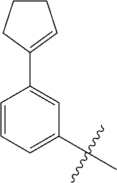 [SMILES: *c1cccc(C2=CCCC2)c1] | 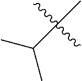 [SMILES: *C(C)C] | 398.2 | DMSO-d₆: δ 8.99 (s, 1H), 8.29-8.26 (m, 2H), 8.21-8.17 (m, 1H), 8.05 (s, 1H), 7.88 (s, 1H), 7.71 (d, J = 7.2 Hz, 1H), 7.55-7.49 (m, 2H), 6.45-6.40 (m, 1H), 5.69-5.64 (m, 1H), 2.75-2.67 (m, 2H), 2.54-2.50 (m, 2H), 2.05-1.97 (m, 2H), 1.64 (d, J = 6.8 Hz, 6H). |
| I-135 | 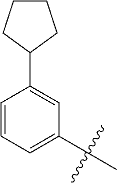 [SMILES: *c1cccc(C2CCCC2)c1] | 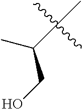 [SMILES: *[C@H](C)CO] | 416.1 | DMSO-d₆: δ 8.88 (s, 1H), 8.28-8.25 (m, 2H), 8.21-8.17 (m, 1H), 7.99 (s, 1H), 7.74 (s, 1H), 7.69-7.67 (m, 1H), 7.46-7.42 (m, 1H), 7.32 (d, J = 7.6 Hz, 1H), 5.66-5.62 (m, 1H), 5.10 (s, 1H), 3.83-3.75 (m, 2H), 3.07-3.03 (m, 1H), 2.12-2.04 (m, 2H), 1.83-1.79 (m, 2H), 1.71-1.64 (m, 7H). |
| I-136 | 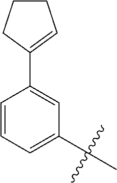 [SMILES: *c1cccc(C2=CCCC2)c1] | 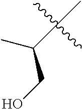 [SMILES: *[C@H](C)CO] | 414.2 | DMSO-d₆: δ 8.88 (s, 1H), 8.35 (s, 1H), 8.28-8.26 (m, 2H), 8.21-8.17 (m, 1H), 8.05 (s, 1H), 7.91 (s, 1H), 7.74 (d, J = 7.2 Hz, 1H), 7.54-7.48 (m, 2H), 6.44 (s, 1H), 5.63-5.60 (m, 1H), 5.11 (s, 1H), 3.85-3.82 (m, 2H), 2.76-2.73 (m, 2H), 2.50-1.97 (m, 2H), 1.93-1.90 (m, 1H), 1.61 (d, J = 6.8 Hz, 3H). |
| I-137 | 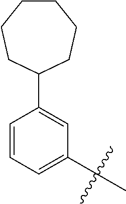 [SMILES: *c1cccc(C2CCCCCC2)c1] | 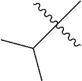 [SMILES: *C(C)C] | 428.2 | DMSO-d₆: δ 9.00 (s, 1H), 8.28-8.25 (m, 2H), 8.21-8.17 (m, 1H), 7.98 (s, 1H), 7.67-7.63 (m, 2H), 7.46-7.42 (m, 1H), 7.28 (d, J = 7.6 Hz, 1H), 5.74- 5.70 (m, 1H), 2.80-2.67 (m, 1H), 1.90- 1.78 (m, 4H), 1.73-1.53 (m, 14H). |
| I-138 | 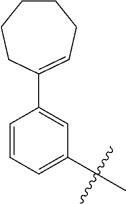 [SMILES: *c1cccc(C2=CCCCCC2)c1] | 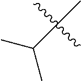 [SMILES: *C(C)C] | 426.2 | DMSO-d₆: δ 8.47 (s, 1H), 8.29-8.25 (m, 2H), 8.21-8.16 (m, 1H), 8.02 (s, 1H), 7.75-7.69 (m, 2H), 7.50-7.45 (m, 1H), 7.37-7.35 (m, 1H), 6.21-6.17 (m, 1H), 5.76-5.67 (m, 1H), 2.64-2.60 (m, 2H), 2.34-2.28 (m, 2H), 1.83-1.79 (m, 2H), 1.65-1.55 (m, 10H). |
| I-139 | 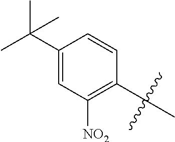 [SMILES: *c1ccc(C(C)(C)C)cc1[N+](=O)[O-]] | 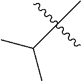 [SMILES: *C(C)C] | 433.1 | CD₃OD-d₄: δ 8.91 (s, 1H), 8.16-8.07 (m, 3H), 7.99 (d, J = 1.8 Hz, 1H), 7.94-7.89 (m, 3H), 5.72-5.68 (m, 1H), 1.61 (d, J = 6.9 Hz, 6H), 1.62 (s, 9H). |
| I-140 | 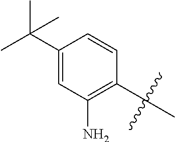 [SMILES: *c1ccc(C(C)(C)C)cc1N] | 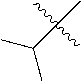 [SMILES: *C(C)C] | 403.2 | CD₃OD-d₄: δ 8.91 (s, 1H), 8.16-8.07 (m, 3H), 7.99 (d, J = 1.8 Hz, 1H), 7.94-7.89 (m, 3H), 5.72-5.68 (m, 1H), 1.61 (d, J = 6.9 Hz, 6H), 1.62 (s, 9H). |
| I-141 | 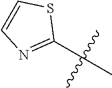 [SMILES: *c1nccs1] | 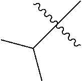 [SMILES: *C(C)C] | 339.1 | DMSO-d₆: δ 8.99 (s, 1H), 8.33-8.31 (m, 1H), 8.28-8.19 (m, 3H), 8.07-8.05 (m, 1H), 7.98 (d, J = 3.2 Hz, 1H), 5.75-5.69 (m, 1H), 1.64 (d, J = 6.8 Hz, 6H). |
| I-142 | 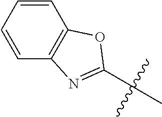 [SMILES: *c1nc2ccccc2o1] | 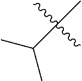 [SMILES: *C(C)C] | 373.1 | CD₃OD-d₄: δ 8.93 (s, 1H), 8.40 (d, J = 7.6 Hz, 1H), 8.33 (d, J = 7.2 Hz, 1H), 8.24-8.20 (m, 2H), 7.82-7.80 (m, 1H), 7.74-7.71 (m, 1H), 7.52-7.46 (m, 2H), 5.90-5.84 (m, 1H), 1.73 (d, J = 6.4 Hz, 6H). |
| I-143 | 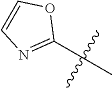 [SMILES: *c1ncco1] | 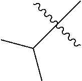 [SMILES: *C(C)C] | 323.1 | DMSO-d₆: δ 9.02 (s, 1H), 8.39 (s, 1H), 8.32-8.21 (m, 3H), 8.15 (s, 1H), 7.54 (s, 1H), 5.65-5.58 (m, 1H), 1.61 (d, J = 6.8 Hz, 6H). |
| I-144 | 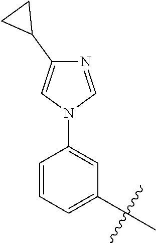 [SMILES: *c1cccc(-n2cnc(C3CC3)c2)c1] | 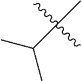 [SMILES: *C(C)C] | 438.3 | DMSO-d₆: δ 8.99 (s, 1H), 8.31-8.28 (m, 2H), 8.23-8.19 (m, 2H), 8.13 (s, 1H), 8.04 (s, 1H), 7.77-7.75 (m, 1H), 7.67 (d, J = 5.2 Hz, 2H), 7.59 (s, 1H), 5.70-5.67 (m, 1H), 1.89-1.85 (m, 1H), 1.62 (d, J = 6.4 Hz, 6H), 0.86-0.81 (m, 2H), 0.74-0.70 (m, 2H). |

### Example 18: Synthesis of Compound II-01

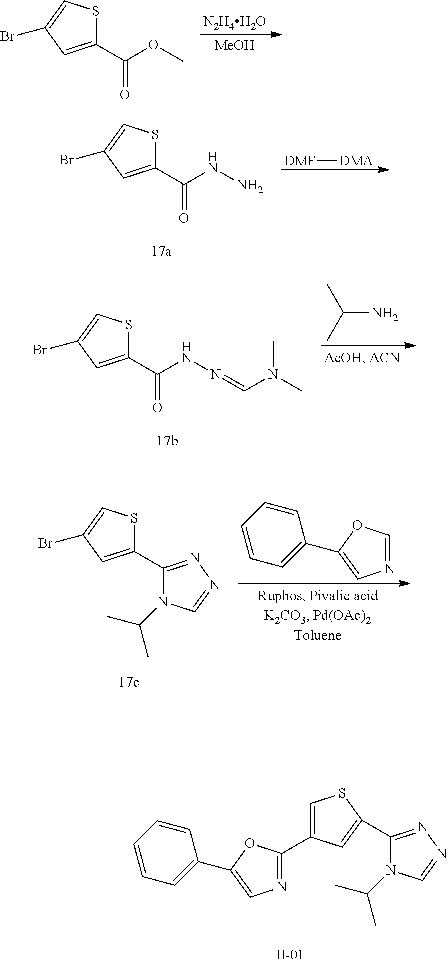

4-Bromothiophene-2-carbohydrazide (Compound 17a): To a solution of methyl 4-bromothiophene-2-carboxylate (5.0 g, 22.7 mmol) in CH₃OH (90 mL) was added N₂H₄.H₂O (4.9 g, 98.2 mmol, 80%) at 25° C. The resulting mixture was stirred at 60° C. for 16 h. After the reaction was completed, the reaction mixture was cooled to room temperature and then filtered. The solid was cooled and dried to afford the title compound (3.0 g, 60%) as a white solid. LCMS (ESI, m/z): [M+H]⁺=221.0.

(E)-N′-(4-Bromothiophene-2-carbonyl)-N,N-dimethylformohydrazonamide (Compound 17b): A solution of Compound 17a (3.0 g, 13.6 mmol) in DMF-DMA (80 mL) was stirred at 80° C. for 16 h. The reaction mixture was concentrated under vacuum. The residue was purified by flash column chromatography with dichloromethane/methanol (9/1, v/v) to afford the title compound (1.1 g, 29%) as a white solid. LCMS (ESI, m/z): [M+H]⁺=276.0.

3-(4-Bromothiophen-2-yl)-4-(propan-2-yl)-4H-1,2,4-triazole (Compound 17c): To a solution of Compound 17b (1.1 g, 4.0 mmol) in CH₃CN (40 mL) and CH₃COOH (10 mL) was added propan-2-amine (1.2 g, 20.0 mmol) at 0° C. The resulting mixture was stirred at 95° C. for 16 h. The pH value of the mixture was adjusted to 8 with NaOH (6 M). The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (9/1, v/v) to afford the title compound (0.7 g, 63%) as a light yellow solid. LCMS (ESI, m/z): [M+H]⁺=272.0.

3-[4-(5-Phenyl-1,3-oxazol-2-yl)thiophen-2-yl]-4-(propan-2-yl)-4H-1,2,4-triazole (Compound II-01): To a solution of Compound 17c (400 mg, 1.48 mmol) in toluene (10 mL) was added 5-phenyl-1,3-oxazole (215 mg, 1.48 mmol), Pd(OAc)₂ (16.5 mg, 0.07 mmol), pivalic acid (60.4 mg, 0.59 mmol), Ruphos (66.6 mg, 0.15 mmol) and K₂CO₃ (613 mg, 4.44 mmol). The mixture was stirred at 110° C. for 16 h under N₂ atmosphere. The resulting mixture was diluted with H₂O and extracted with ethyl acetate. The organic layer was washed with brine, dried over anhydrous sodium sulfate and filtered. The filtrate was concentrated under vacuum. The residue was purified by flash column chromatography with dichloromethane/methanol (9/1, v/v) and then purified by Prep-HPLC with the following conditions: Column: YMC-Actus Triart C18 Column 30×250 mm Sum; Mobile Phase A:Water (10 mmol/L NH₄HCO₃), Mobile Phase B: ACN; Flow rate: 60 mL/min; Gradient: 43% B to 73% B in 7 min; 254, 220 nm; Rt: 6.13 min to afford the title compound (3.1 mg, 0.6%) as a white solid. LCMS (ESI, m/z): [M+H]⁺=337.1. ¹H NMR (400 MHz, DMSO-d₆, ppm): δ 8.91 (s, 1H), 8.52 (d, J=1.2 Hz, 1H), 7.99 (d, J=1.2 Hz, 1H), 7.87-7.84 (m, 3H), 7.54-7.51 (m, 2H), 7.43-7.37 (m, 1H), 4.80-4.73 (m, 1H), 1.56-1.50 (m, 6H).

### Example 19: Biological Activity of Compounds

### Assay Protocol

Reagents: Recombinant human ASK1 kinase was from Invitrogen Inc. HTRF KinEASE™-STK S3 kit was obtained from Cisbio (Bedford, Mass.). All other reagents were of the highest grade commercially available.

Assay: The assay measures the phosphorylation level of a biotinylatd peptide substrate by the ASK1 kinase using HTRF detection. ASK1 kinase assay is based on HTRF KinEASE™-STK manual from Cisbio. Test compound, 1 uM STK-substrate-biotin, 3 nM of ASK1 kinase are incubated with 1*Kinase buffer with 5 mM MgCl2 and 1 mM DTT. 250 nM Sa-XL 665(4*) and STK-antibody-Cryptate work solution are added to stop the reaction and phosphorylated peptide substrate is detected using Envision 2104 Multilabeled reader from PerkinElmer. The fluorescence is measured at 615 nm (Cryptate) and 665 nm (XL665) and a ratio of 665 nm/615 nm is calculated for each well. The resulting TR-FRET level (a ratio of 665 nm/615 nm) is proportional to the phosphorylation level of substrate.

TABLE 2
BIOLOGY DATA

| | EC50 |
|---|---|
| Code | (nM) |
| I-002 | A |
| I-017 | B |
| I-004 | A |
| I-018 | A |
| I-019 | B |
| I-015 | A |
| I-021 | B |
| I-024 | A |
| I-025 | A |
| I-026 | B |
| I-027 | A |
| I-013 | C |
| I-028 | B |
| I-029 | A |
| I-030 | A |
| I-031 | A |
| I-032 | A |
| I-033 | B |
| I-034 | B |
| I-035 | B |
| I-036 | B |
| I-037 | B |
| I-038 | A |
| I-039 | B |
| I-040 | A |
| I-041 | A |
| I-001 | A |
| I-042 | A |
| I-043 | B |
| I-044 | B |
| I-045 | B |
| I-003 | B |
| I-049 | B |
| I-050 | A |
| I-051 | A |
| I-052 | A |
| I-053 | A |
| I-057 | A |
| I-058 | A |
| I-059 | A |
| I-060 | A |
| I-061 | A |
| I-062 | A |
| I-063 | A |
| I-064 | A |
| I-065 | A |
| I-066 | A |
| I-067 | A |
| I-068 | A |
| I-070 | B |
| I-071 | B |
| I-072 | A |
| I-073 | A |
| I-074 | A |
| I-076 | A |
| I-077 | A |
| I-078 | A |
| I-079 | A |
| I-080 | A |
| I-081 | B |
| I-082 | B |
| I-083 | B |
| I-084 | A |
| I-085 | B |
| I-086 | B |
| I-087 | B |
| I-089 | B |
| I-090 | B |
| I-091 | B |
| I-092 | A |
| I-093 | B |
| I-008 | B |
| I-094 | A |
| I-009 | B |
| I-095 | B |
| I-006 | B |
| I-096 | A |
| I-097 | A |
| I-098 | A |
| I-099 | A |
| I-100 | A |
| I-101 | B |
| I-102 | A |
| I-103 | B |
| I-104 | B |
| I-105 | B |
| I-106 | B |
| I-107 | A |
| I-108 | A |
| I-109 | A |
| I-110 | A |
| I-111 | A |
| I-112 | C |
| I-113 | A |
| I-114 | A |
| I-115 | A |
| I-116 | A |
| I-010 | A |
| I-117 | A |
| I-118 | A |
| I-012 | A |
| I-119 | A |
| I-011 | A |
| I-120 | B |
| I-121 | B |
| I-122 | B |
| I-123 | A |
| I-124 | B |
| I-125 | A |
| I-126 | A |
| I-127 | A |
| I-128 | A |
| I-005 | A |
| I-016 | B |
| I-129 | A |
| I-130 | B |
| I-131 | C |
| I-132 | B |
| I-133 | B |
| I-134 | B |
| I-135 | A |
| I-136 | A |
| I-138 | B |
| I-141 | B |
| I-142 | B |
| I-144 | A |
| II-01 | B |
| — | |
| — | |

A: EC₅₀ ≤ 50 nM;
B: 50 nM < EC₅₀ ≤ 500 nM;
C: 500 nM < EC₅₀ ≤ 5000 nM.

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A compound of Formula I: 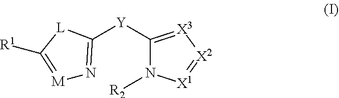
and pharmaceutically acceptable salts thereof;
wherein
L is O or S;
M is CH or N;
X¹ is CH or N;
X² is CH or N;
X³ is CH or N;
Y is a substituted or unsubstituted phenyl or a 5- or 6-member heteroaryl group;
R¹ is a substituted or unsubstituted cycloalkyl, aryl or heteroaryl group; and
R² is substituted or unsubstituted alkyl, cycloalkyl, cycloalkylalkyl, aryl, or aralkyl group.

2. The compound of claim 1 of Formula IA: 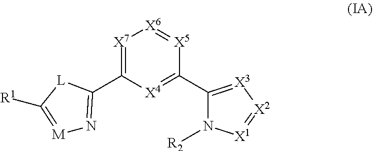
and pharmaceutically acceptable salts thereof,
wherein
X⁴ is CR⁴ or N;
X⁵ is CR⁵ or N;
X⁶ is CR⁶ or N;
X⁷ is CR⁷ or N; and
R⁴, R⁵, R⁶, and R⁷ are independently H, halo, OH, NO₂, CN, COOH, C(O)O(alkyl), C(O)O(aralkyl), C(O)O(alkenyl), C(O)(alkyl), NH₂, C(O)NH₂, NH(alkyl), N(alkyl)₂, thioalkyl, haloalkyl, alkoxy, haloalkoxy, hydroxyalkyl, or a substituted or unsubstituted alkyl or cycloalkyl group.

3. The compound of claim 1 wherein X¹ is CH.

4. The compound of claim 1 wherein X² is N.

5. The compound of claim 1 wherein X³ is N.

6. The compound of claim 1 having the Formula IB: 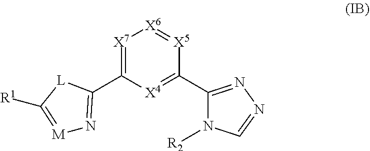
and pharmaceutically acceptable salts thereof.

7. The compound of claim 2 wherein X⁴ is N.

8. The compound of claim 2 wherein X⁵ is CR⁵.

9. (canceled)

10. The compound of claim 2 wherein X⁶ is CR⁶.

11. (canceled)

12. The compound of claim 2 wherein X⁷ is CR⁷.

13. (canceled)

14. The compound of claim 1 having the Formula IC: 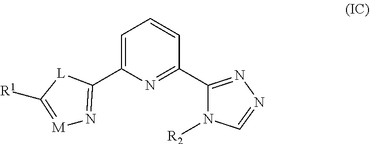
and pharmaceutically acceptable salts thereof.

15. The compound of claim 1 wherein L is O.

16. The compound of claim 1 wherein M is CH.

17. The compound of claim 1 having the Formula ID: 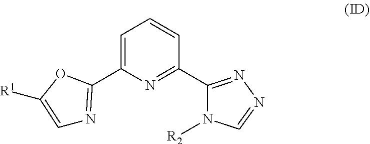
and pharmaceutically acceptable salts thereof.

18. The compound of claim 1 wherein R¹ is a phenyl, naphthyl, tetrahydronaphthyl, cyclohexyl, pyridinyl, 2,3-dihydrobenzo[b][1,4]dioxinyl, quinolinyl, isoquinolinyl, pyrazinyl, pyrimidinyl, or oxazolyl group, optionally substituted with one or more substituents selected from the group consisting of F, Cl, Br, I, OH, CN, COOH, C(O)OR^{a}, C(O)R^{b}, C(O)NR^{c}R^{d}, NO₂, C(O)NH₂, NR^{e}R^{f}, SO₂NR^{g}R^{h}, alkyl, thioalkyl, haloalkyl, alkoxy, alkoxyalkoxy, haloalkoxy, hydroxyalkyl, cycloalkyl, cycloalkenyl, SO₂R^{j}, phenyl, pyrrolinyl, N-Boc-pyrrolinyl, aminopyrrolidinyl, N-Boc-aminopyrrolidinyl, pyrrolidinyl, imidazolyl, cyclopropyl-imidazolyl, oxazolyl, benzoxazolyl, thiazolyl, tetrahydro-2H-pyranyl, morpholinyl, N-alkylmorpholinyl, morpholinylalkoxy, piperidinyl, 4-morpholinyl-piperidinyl, piperazinyl, N-alkylpiperazinyl, N-cycloalkylpiperazinyl, N-sulfonylalkyl, azabicyclo-[3, 2, 1]-octanyl, and pyridinyl, wherein
R^{a}, R^{b}, R^{c}, R^{d}, R^{e}, R^{f}, R^{g}, and R^{h} are independently H or substituted or unsubstituted alkyl, alkenyl, cycloalkyl, aryl, aralkyl, heterocyclyl, heterocyclylalkyl, heteroaryl, or heteroaralkyl; and
R^{j} is substituted or unsubstituted alkyl, alkenyl, cycloalkyl, aryl, aralkyl, heterocyclyl, heterocyclylalkyl, heteroaryl, or heteroaralkyl.

19. The compound of claim 18, wherein the R¹ group is substituted with 1, 2 or 3 substituents.

20. The compound of claim 1 wherein R² is a phenyl(C₁-C₆ alkyl), C₁-C₆ alkyl or C₃-C₆ cycloalkyl group optionally substituted with one or more substituents selected from the group consisting of F, CF₃, OH, NH₂, OCH₃.

21. The compound of claim 1, wherein Y is a thiophenyl group.

22. A composition comprising a compound of claim 1 and a pharmaceutically acceptable carrier.

23. A pharmaceutical composition comprising an effective amount of the compound of claim 1 for treating an ASK1-mediated disorder or condition.

24. The pharmaceutical composition of claim 23 wherein the disorder or condition is selected from the group consisting of fibrotic diseases, acute and chronic liver diseases, kidney diseases, autoimmune disorders, inflammatory diseases, cardiovascular diseases, diabetes, diabetic nephropathy, cardio-renal diseases, and neurodegenerative diseases.

25-26. (canceled)

27. A method of treating a disease or disorder comprising administering an effective amount of a compound of claim 1 or an effective amount of a composition of claim 22 to a subject suffering from the disease or disorder mediated by ASK1.

28. The method of claim 27, wherein the disorder or condition is selected from the group consisting of fibrotic diseases, acute and chronic liver diseases, kidney diseases, autoimmune disorders, inflammatory diseases, cardiovascular diseases, diabetes, diabetic nephropathy, cardio-renal diseases, and neurodegenerative diseases.

29. (canceled)

30. A method comprising inhibiting ASK1 by contacting ASK1 with an effective amount of a compound of claim 1.
